(19) United States
(12) Patent Application Publication
(10) Pub. No.: US 2026/0243280 A1
(43) Pub. Date: Aug. 20, 2026

(54) MULTI-FUNCTIONING LOCKING MECHANISM OF A HOLD OPEN ROD

(71) Applicant: MarathonNorco Aerospace, Inc., Waco, TX (US)

(72) Inventor: Steven Pape, Riesel, TX (US)

(21) Appl. No.: 19/539,811

(22) Filed: Feb. 13, 2026

Related U.S. Application Data
(60) Provisional application No. 63/758,505, filed on Feb. 14, 2025.

(51) Int. Cl. F15B 15/26; A61B 17/70; E21B 17/046

(52) U.S. Cl. CPC F15B 15/261; A61B 17/7014; E21B 17/046

(57) ABSTRACT

A hold open rod that includes a locking collar that may be configured to operate in a first unlocked mode and a second unlocked mode in which the hold open rod is adjustable in a different manner than the first unlocked mode. For example, when in the first unlocked mode, the locking collar may be configured to lock the hold open rod when the hold open rod is compressed to an intermediately compressed configuration. When in the second unlocked mode, the locking collar may be configured allow the hold open rod to compress to the intermediately compressed configuration and continue compressing without locking the hold open rod, thereby bypassing locking of the hold open rod when in the intermediately compressed configuration.

### CROSS-REFERENCE TO RELATED APPLICATIONS

This application claims the benefit from U.S. Provisional Application No. 63/758,505 filed on Feb. 14, 2025, which is hereby incorporated by reference in its entirety for all purposes as if fully set forth herein.

### TECHNICAL FIELD

The disclosure relates to locking mechanisms. More particularly, this disclosure relates to a lock mechanism that may be implemented as part of a hold open rod, such as those utilized on vehicles (e.g., aircraft cowling or nacelle).

### BACKGROUND

Some automotive and aviation vehicles include one or more hold open rods. A hold open rod may be used to hold a door or hatch open by supporting the weight of the door or hatch (e.g., an aircraft cowling or nacelle door). Such may provide for easier access by ground maintenance personnel, who can use the hold open rod to maintain the door in an open position.

Hold open rods may include two cylindrical and telescoping tubes, in which an inner tube is located inside of an outer tube, and a locking mechanism that is configured to lock the outer tube and the inner tube in an extended position, thereby preventing the inner tube from axially retracting into the outer tube. Thus, the locking mechanism may prevent the outer tube and the inner tube from axially compressing together when the door is in the open position.

Some doors, however, are heavy and can be shaped in a manner that is difficult for maintenance personnel to safely and easily close when unlocking the locking mechanism, especially when only a single maintenance crew member is available. For example, some doors may require the maintenance crew member to climb onto a ladder to unlock the locking mechanism and hold the door as it closes, but require the maintenance crew member to bend too low or unsafely get off of the ladder while holding the door to lower the door to its closed position. Thus, the locking mechanisms of some doors may require more than a single maintenance crew member to safely unlock the respective locking mechanism and close the respective door.

Accordingly, it is desirable to have a simple lock mechanism that is able to provide the maintenance crew member with the ability to lock the hold open rod in an intermediate position, prior to the door being fully closed.

### SUMMARY

The present application provides for a hold open rod that includes a locking collar that may be configured to operate in a first unlocked mode and a second unlocked mode in which the hold open rod is adjustable in a different manner than the first unlocked mode. For example, when in the first unlocked mode, the locking collar may be configured to lock the hold open rod when the hold open rod is compressed to an intermediately compressed configuration. When in the second unlocked mode, the locking collar may be configured allow the hold open rod to compress to the intermediately compressed configuration and continue compressing without locking the hold open rod, thereby bypassing locking of the hold open rod when in the intermediately compressed configuration.

The hold open rod may have more than two modes. For example, the inner tube may include three or more paths that a locking pin of the locking collar can translate over.

The hold open rod may be compressed or extended such that the locking collar reaches a first locking portion of the inner tube. While in a first mode the locking collar may be configured to engage the first locking portion such that the hold open rod is locked. While in a second mode the locking collar may be configured to bypass the first locking portion such that the hold open rod remains unlocked.

Locking the hold open rod in the intermediately compressed configuration, for example, may provide the maintenance crew member an opportunity to reposition themselves (e.g., stepping lower or off of a ladder) or perform another task before closing the door, without requiring another maintenance crew member to ensure safety. For example, for large cowlings, a single operator can close the cowling halfway while standing on a ladder. The hold open rod may be in the first mode to lock the hold open rod in the intermediately compressed configuration such that the cowling remains halfway closed. The operator may then adjust their ladder or their operating position, unlock the locking collar again, and continue closing the cowling The operator (or another taller operator) may choose to close the cowling while the hold open rod is in the second mode, in which the operator may fully close the cowling (from its fully open position) without stopping to unlock the hold open rod at the intermediately compressed configuration. For example, if the operator is tall or dexterous enough to safely close the cowling without needing the hold open rod to support the cowling halfway open, the operator may choose to operate the hold open rod in the second mode when closing the cowling from its fully open position.

Thus, the hold open rod may provide for improved safety and ergonomics while also providing operators with a choice as to how quickly to close a cowling. For example, the hold open rod may support the cowling halfway open when closing the cowling may be difficult for the operator. Also, the hold open rod may allow the operator (or multiple operators working together) the option to quickly close the cowling without needing to unlock the hold open rod again when the cowling is halfway closed, thereby saving the operator's time.

According to an embodiment of the present disclosure, a hold open rod comprises an outer tube, an inner tube, and a locking collar. The inner tube and the outer tube may be configured to translate relative to one another when the hold open rod is unlocked such that the inner tube translates within the outer tube along a longitudinal axis. The locking collar may be configured to lock and unlock the outer tube and the inner tube. The locking collar may be movable to a first unlocked position and a second unlocked position, where the hold open rod is configured to be in a first mode when the locking collar is in the first unlocked position, and where the hold open rod is configured to be in a second mode when the locking collar is in the second unlocked position. When in the first mode, the locking collar may be configured to lock the outer tube and the inner tube when the outer tube translates relative to the inner tube in a first axial direction such that the locking collar moves to a first locking portion of the inner tube and prevents the outer tube from translating relative to the inner tube in the first axial direction beyond a predetermined position. When in the second mode, the locking collar may be configured to not lock the outer tube and the inner tube when the outer tube translates relative to the inner tube in the first axial direction to the first locking portion of the inner tube such that the locking collar allows the outer tube to translate relative to the inner tube to and beyond the predetermined position.

According to another embodiment of the present disclosure, a method of operating a hold open rod comprises moving a locking collar, which may be movable to a first unlocked position and movable to a second unlocked position, to the second unlocked position. The hold open rod may be in a first mode when the locking collar is in the first unlocked position, and the hold open rod may be in a second mode when the locking collar is in the second unlocked position. The method may comprise translating, while the hold open rod is in the second mode, the outer tube relative to the inner tube in a first axial direction along a longitudinal axis such that the locking collar moves toward a first locking portion of the inner tube, wherein the hold open rod remains unlocked such that the outer tube translates relative to the inner tube in the first axial direction to and beyond a predetermined position. The locking collar may be configured to, when in the first mode, lock the outer tube and the inner tube when the outer tube translates relative to the inner tube in the first axial direction such that the locking collar moves to the first locking portion of the inner tube and prevents the outer tube from translating relative to the inner tube in the first axial direction beyond the predetermined position.

There has thus been outlined, rather broadly, certain aspects of the disclosure in order that the detailed description thereof herein may be better understood, and in order that the present contribution to the art may be better appreciated. There are, of course, additional aspects of the disclosure that will be described below and which will form the subject matter of the claims appended hereto.

In this respect, before explaining at least one aspect of the disclosure in detail, it is to be understood that the disclosure is not limited in its application to the details of construction and to the arrangements of the components set forth in the following description or illustrated in the drawings. The disclosure is capable of aspects in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the disclosure. It is important, therefore, that the claims be regarded as including such equivalent constructions insofar as they do not depart from the spirit and scope of the disclosure.

### BRIEF DESCRIPTION OF THE DRAWINGS

The foregoing summary, as well as the following detailed description of illustrative embodiments of the hold open rod of the present application, will be better understood when read in conjunction with the appended drawings. For the purposes of illustrating the hold open rod of the present application, there is shown in the drawings illustrative embodiments. It should be understood, however, that the application is not limited to the precise arrangements and instrumentalities shown. In the drawings:

FIG. 1 is a partial cross-sectional view of an aircraft structure implementing a hold open rod according to the disclosure;

FIG. 2 is an oblique view of the hold open rod of FIG. 1, shown in an extended configuration;

FIG. 3 is a cross-sectional oblique view of the hold open rod of FIG. 2, shown in the extended configuration;

FIG. 4A is a side cross-sectional view of the hold open rod of FIG. 3;

FIG. 4B is a side cross-sectional view the hold open rod of FIG. 4A in including a locking collar that is locking an inner tube and an outer tube of the hold open rod;

FIG. 5 is a cross-sectional oblique view of the hold open rod of FIG. 4B;

FIG. 6 is another cross-sectional oblique view of the hold open rod of FIG. 4B;

FIG. 7 is a cross-sectional oblique view of the hold open rod of FIG. 4B, in which the locking collar is in a first unlocked position;

FIG. 8 is an oblique view of the hold open rod of FIG. 7, in which a locking collar body of the locking collar is removed so that locking dogs and a locking pin of the locking collar are visible;

FIG. 9 is another oblique view of the hold open rod of FIG. 8, in which a guiding pin of the locking collar and a guiding channel of the outer tube are shown;

FIG. 10A is another oblique view of the hold open rod of FIG. 8, in which the locking pin is removed so that an unlocking channel of the outer tube is more visible;

FIG. 10B an oblique view of the inner tube of the hold open rod of FIG. 2, including a structure mount;

FIG. 11 is a cross-sectional oblique view of the hold open rod of FIG. 4B, in which the locking collar is in a second unlocked position;

FIG. 12 is an oblique view of the hold open rod of FIG. 11, in which the locking collar body is removed so that the locking dogs and the locking pin are visible;

FIG. 13 is an oblique view of the hold open rod of FIG. 1, shown in an intermediately extended configuration, in which the locking collar is locked;

FIG. 14 is another oblique view of the hold open rod of FIG. 13, in which the locking collar body is removed so that the unlocking channel and the locking pin are visible;

FIG. 15A is an oblique view of the hold open rod of FIG. 14, in which a second elongated tubular body of the outer tube is removed so that the locking dogs and a first locking portion of the inner tube are visible;

FIG. 15B is an oblique view of the hold open rod of FIG. 1, shown in a compressed configuration, in which the locking collar is in a first unlocked position;

FIG. 15C is an oblique view of the hold open rod of FIG. 15B, in which the locking collar body is removed so that the unlocking channel and the locking pin are visible;

FIG. 16A is an oblique view of the hold open rod of FIG. 1, in which the hold open rod is extended to a stowed configuration;

FIG. 16B is another oblique view of the hold open rod of FIG. 16A, in which the locking collar body is removed so that the unlocking channel and the locking pin are visible;

FIG. 16C is an oblique view of the hold open rod of FIG. 16B, in which the second elongated tubular body of the outer tube is removed so that the locking dogs and the first locking portion of the inner tube are visible;

FIG. 17A is an oblique view of the hold open rod of FIG. 1, in which the locking collar is in the second unlocked position and the hold open rod is slightly extended from the intermediately extended configuration;

FIG. 17B is an oblique view of the hold open rod of FIG. 17A, in which the locking collar body is removed so that the unlocking channel and the locking pin are visible;

FIG. 17C is an oblique view of the hold open rod of FIG. 17A, in which the second elongated tubular body of the outer tube is removed so that the locking dogs and the first locking portion are visible;

FIG. 17D is an oblique view of the hold open rod of FIG. 17B, in which the hold open rod is slightly further compressed and the locking pin is being unpinned by the inner tube;

FIG. 17E is an oblique view of the hold open rod of FIG. 17D, in which the second elongated tubular body of the outer tube is removed so that the locking dogs and the first locking portion are visible;

FIG. 18A is a cross-sectional oblique view of the hold open rod of FIG. 1, in which the locking collar is in the first unlocked position and the locking pin will be unpinned by the inner tube when the hold open rod is slightly further extended;

FIG. 18B is an oblique view of the hold open rod of FIG. 18A, in which the locking collar body and the second tubular body are removed so that the locking dogs, the locking pin, and the first locking portion are visible;

FIG. 19A is an oblique view of the hold open rod of FIG. 1, in which the locking collar body, the locking dogs, and the locking pin are removed and each end of a biasing member of the hold open rod is visible; and

FIG. 19B is an oblique view of the locking collar body of the hold open rod of FIG. 1, in which a biasing member channel is visible.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about” or “substantially” it will be understood that the particular value forms other embodiments including plus or minus 10% of the range or value provided. All ranges are inclusive and combinable.

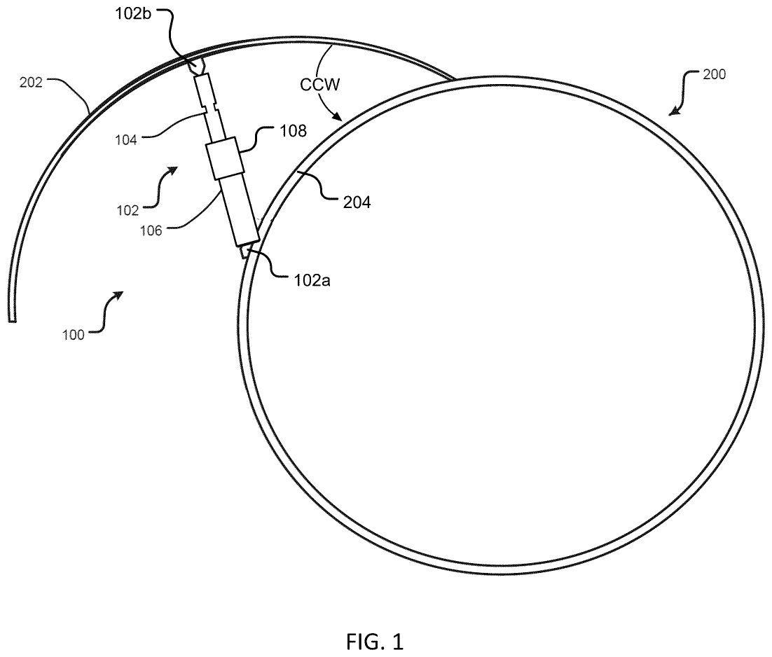

Referring to FIG. 1, a partial cross-sectional view of an aircraft structure 200 implementing a hold open rod assembly 100 is schematically represented. The hold open rod assembly 100 may include a hold open rod 102 (also referred to as a strut), which may include an inner tube 104 and an outer tube 106. The inner tube 104 and the outer tube 106 may be in axial alignment and translatable relative to one another in a telescoping manner when the hold open rod assembly 100 is in an unlocked activated state. The hold open rod 102 may be operably coupled to a door component 202 (e.g., an aircraft cowling or nacelle door) of the aircraft structure 200 such that the inner tube 104 is configured to translate relative to the outer tube 106 when the door component 202 moves (e.g., opens or closes). Likewise, the outer tube 106 is configured to translate relative to the inner tube 104 when the door component 202 moves. For example, the outer tube 106 may remain stationary while the door component 202 moves in three-dimensional space such that the outer tube 106 translates relative to the inner tube 104 due to the movement in three-dimensional space of the inner tube 104 with the door component 202.

A door mount 102a and a structure mount 102b of the hold open rod assembly 100 may be respectively coupled to the door component 202 and a housing 204 of the aircraft structure 200 (e.g., a frame or other support structure of the aircraft structure). The door mount 102a and/or the structure mount 102b may be configured to couple the hold open rod 102 to the door component 202 and the housing 204, respectively, such that the hold open rod 102 is pivotable relative to the door component 202 and the housing 204 as the door component 202 moves. For example, the door component 202 may be configured to pivot relative to the housing 204 to open and to close, and thus the hold open rod 102 may be configured to pivot relative to the door component 202 and the housing 204 when the door component 202 opens and when the door component 202 closes. In an alternative embodiment, the outer tube is coupled to the door component and the inner tube is coupled to the housing.

The hold open rod 102 may be configured such that when the door component 202 closes from the open position represented in FIG. 1, the hold open rod 102 may compress to a minimum length (also referred to as a fully compressed configuration) along a longitudinal axis X when the door component 202 is in a partially closed position. The hold open rod 102 may also be configured such that when the door component 202 closes from the partially closed position to a fully closed position, the hold open rod 102 may expand slightly to a partially extended length (also referred to as a stowed configuration) that may be anywhere from 50% to 75% of the fully extended length of the hold open rod 102. In an embodiment, the door component may be fully closed when the hold open rod is in its fully compressed configuration.

When the door component 202 closes from the partially closed position to the fully closed position, the door component 202 may pivot in a counterclockwise direction (CCW) such that the door component 202 forms a contiguous outer surface with the housing 204.

In an embodiment, the hold open rod assembly 100 may be operably coupled to components of another vehicle, such as a door of an automobile, or a non-mobile structure, such as a stationary building, or the like.

Certain terminology is used in the following description for convenience only and is not limiting. The words “front,” “rear”, “right”, “left”, “lower” and “upper” designate directions in the drawings to which reference is made. The words “inner”, “internal”, and “interior” refer to directions towards the geometric center of the hold open rod 102, while the words “outer”, “external”, and “exterior” refer to directions away from the geometric center of the hold open rod 102. The terminology includes the above-listed words, derivatives thereof and words of similar import.

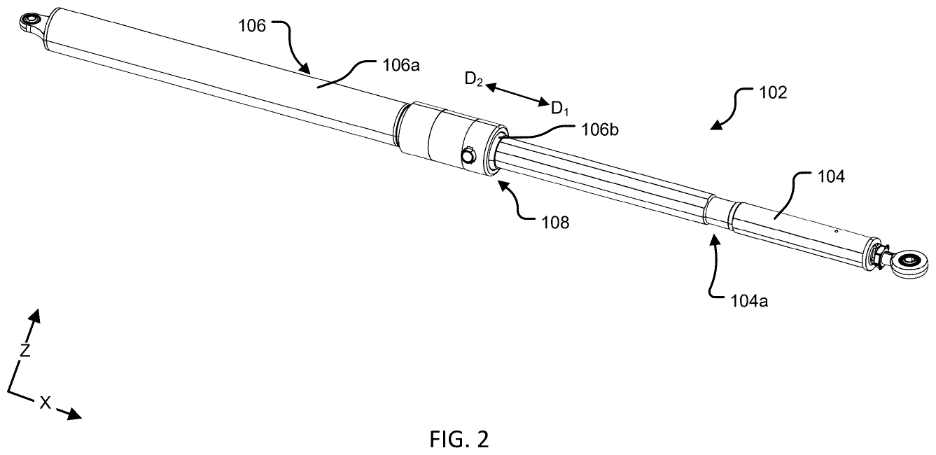

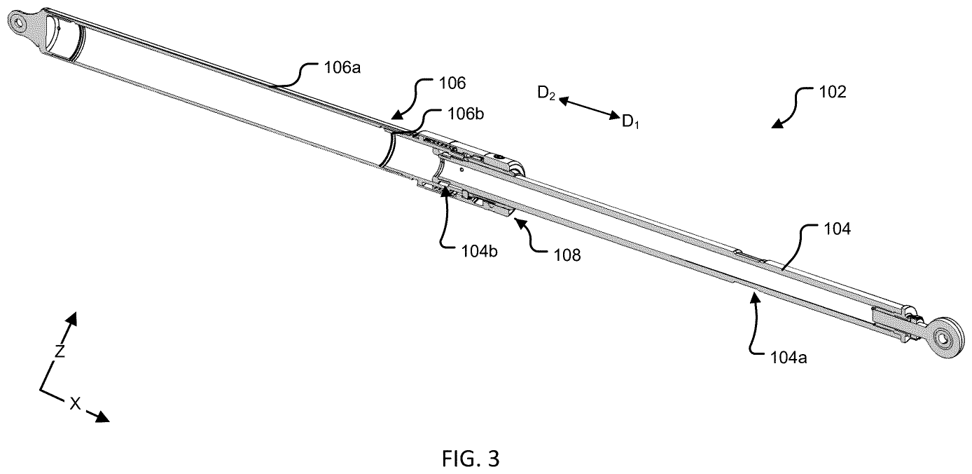

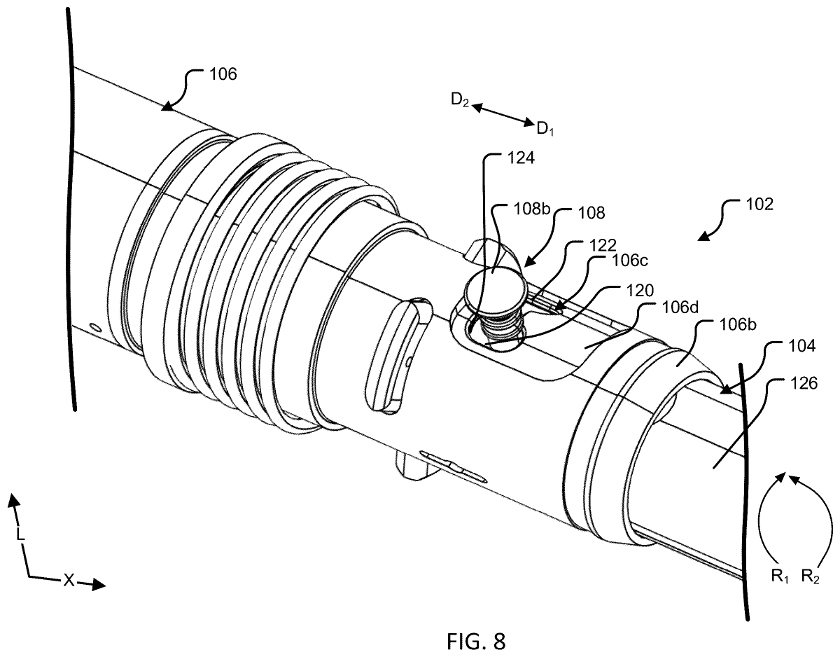

Turning to FIGS. 2-4B, the hold open rod 102 is described herein as extending horizontally along a longitudinal axis “X” and a lateral axis “L” (see e.g., FIG. 8), and vertically along a vertical axis “Z”. The longitudinal axis X can be at least substantially perpendicular to each of the lateral axis L and the vertical axis Z. The lateral axis L can be at least substantially perpendicular to each of the longitudinal axis X and the vertical axis Z. The vertical axis Z can be at least substantially perpendicular to each of the longitudinal and lateral axis X, L. Unless otherwise specified herein, the terms “longitudinal,” “lateral,” and “vertical” are used to describe the orthogonal directional components of various hold open rod and hold open rod component axes regardless of the actual orientation of the hold open rod 102 relative to a vehicle or structure that the hold open rod 102 is installed as part of. Additionally, it should be appreciated that while the longitudinal and later directions X, L are illustrated as extending along and defining a horizontal plane (also referred to herein as a “longitudinal-lateral plane”), and that the vertical axis is illustrated as extending along a vertical plane (such as either a “vertical-longitudinal plane” or a “vertical-lateral plane,” as respectively referred to herein), the planes that encompass the various directions may differ during use. For instance, when the hold open rod 102 is installed as part of a vehicle or structure, the longitudinal axis X may extend generally along a direction of gravity and the lateral axis L and the vertical axis Z may extend generally perpendicularly to the direction of gravity, when the vehicle or structure is in an upright position relative to gravity. Accordingly, the directional terms “longitudinal,” “vertical,” “lateral,” and “horizontal” may be used to describe the hold open rod 102 and its components as illustrated merely for the purposes of clarity and illustration. With the foregoing in mind, the terms “expand” and “expansion,” when used in reference to the hold open rod 102, refer to expansion along the longitudinal axis X.

The hold open rod 102, including the inner tube 104 and/or the outer tube 106, may be made from a metallic material, or a polyamide material, including aliphatic polyamides, polyphthalamides, aromatic polyamides, carbon fiberglass, or another type of thermoplastic compound. Alternative and/or additional materials including composite materials are contemplated as well.

The inner tube 104 and the outer tube 106 may be configured to translate relative to one another while the inner tube 104 is within the outer tube 106. The outer tube 106 may be configured to translate relative to the inner tube 104 in a first axial direction D1 along the longitudinal axis X. The inner tube 104 may be configured to translate relative to the outer tube 106 in a second axial direction D2 along the longitudinal axis X that is opposite the first direction D1. For example, the longitudinal axis X may be concentric with a central axis of the inner tube 104 and/or the outer tube 106.

As discussed above, the inner tube 104 may be cylindrical and the outer tube 106 may be cylindrical, such that the inner tube 104 is slidable within the outer tube 106. In an alternative embodiment, inner tube and the outer tube 106 may have another cross-sectional shape, such as a square cross-section, a hexagonal cross-section, or the like.

The hold open rod 102 may include a locking collar 108 that is configured to lock and unlock the outer tube 106 and the inner tube 104. The inner tube 104 and the outer tube 106 may be configured to translate relative to one another when the hold open rod 102 is unlocked. For example, when unlocked the inner tube 104 may be translatable within the outer tube 106 along the longitudinal axis X. The hold open rod 102 may be configured to extend and/or compress when unlocked.

When the locking collar 108 is locked, the hold open rod 102 may be unable to compress and/or extend. For example, when locked the outer tube 106 may be unable to translate relative to the inner tube 104 in a first direction D1. The inner tube 104 may be unable to translate relative to the outer tube 106 in a second direction D2 that is opposite the first direction D1.

The hold open rod 102 may be configured to only extend a limited distance when locked. For example, the hold open rod 102 may be configured to extend 5% of its total extended length when locked in the extended configuration. In an embodiment, the hold open rod 102 may be configured to extend at most 10%, 5%, 4%, 3%, 2%, 1%, 0.5%, 0.4%, 0.3%, 0.2%, or 0.1% of its total extended length when locked in the extended configuration. In an embodiment, the hold open rod may be configured to not extend when locked, or only extended the limited distance, when in the extended configuration or a more compressed configuration. In an alternative embodiment, the hold open rod may be configured to not compress when locked and may be configured to extend to a fully extended configuration when locked.

The hold open rod 102 may be in the extended configuration when the hold open rod 102 is in a fully extended configuration such that the inner tube 104 and the outer tube 106 together have a maximum length along the longitudinal axis X. In an embodiment, the hold open rod may be in the extended configuration when the hold open rod is in a near fully extended configuration, such that the inner tube and the outer tube together have a near maximum length. For example, the length may be within 20%, 10%, 5%, 4%, 3%, 2%, or 1% of the maximum length of the inner tube and the outer tube together.

The outer tube 106 may include a first elongated tubular body 106a and a second elongated tubular body 106b that coupled to the first elongated tubular body 106a. For example, the second elongated tubular body 106b may be coupled to an inner end of the first elongated tubular body 106a. In an embodiment, the outer tube is a single monolithic body.

The first elongated tubular body 106a may circumscribe a majority of the inner tube 104 when the hold open rod 102 is in a compressed configuration. The outer tube 106 may include a second elongated tubular body 106b that is coupled an inner end of the first elongated tubular body 106a.

The second elongated tubular body 106b may circumscribe a portion of the inner tube 104 that is offset from the majority of the inner tube 104 when the hold open rod 102 is in the compressed configuration. The second elongated tubular body 106b may have an axial length that is less than the length of the first elongated tubular body 106a. For example, the second elongated tubular body 106b may have an axial length that is 15% of an axial length of the inner tube 104. In an embodiment, the second elongated tubular body 106b may have an axial length that is less than 50%, 40%, 30%, 20%, 15%, 10%, 5%, 4%, 3%, 2%, or 1% of an axial length of the inner tube 104.

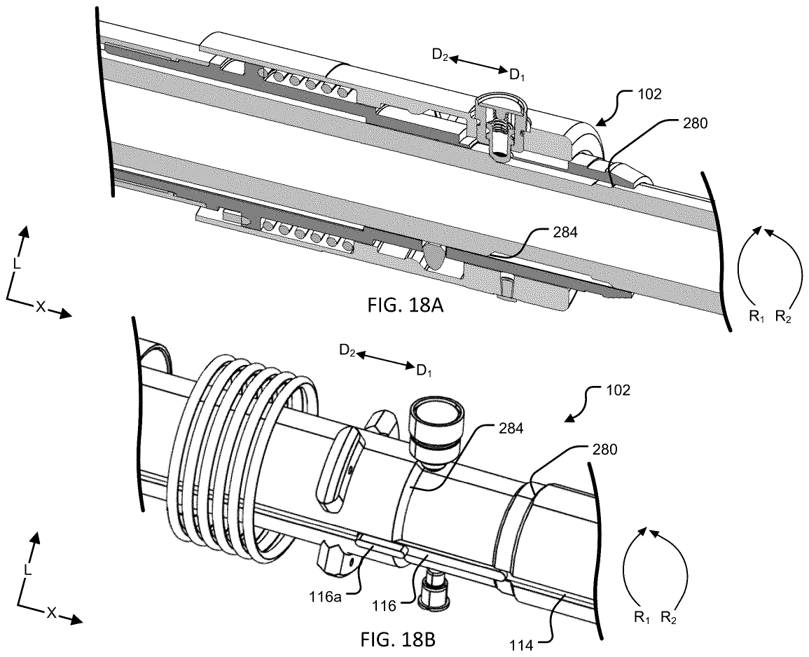

The inner tube 104 and the outer tube 106 may be translatably coupled to one another. For example, the inner tube 104 may include an axially extending groove 114 (see e.g., FIG. 18B) and the hold open rod 102 may include a key 116 (see e.g., FIG. 18B) that is coupled to the outer tube 106 and configured to translate axially within the axially extending groove 114. For example, the key 116 may include a radially outwardly extending tab 116a that is configured to be received in a corresponding groove (not shown) of the outer tube 106 such that the key 116 is axially fixed relative to the outer tube 106. In an embodiment, the hold open rod includes another configuration of a key and groove connection between the inner tube and the outer tube, or another suitable connection that restrains relative movement between the inner tube 104 and the outer tube 106 to translational movement. For example, the outer tube 106 may include a radially inwardly extending key (not shown) that is configured to translate along the axially extending groove 114. In an alternative embodiment, the inner tube may include a key that is translatable within a radially inwardly facing axially extending groove of the outer tube.

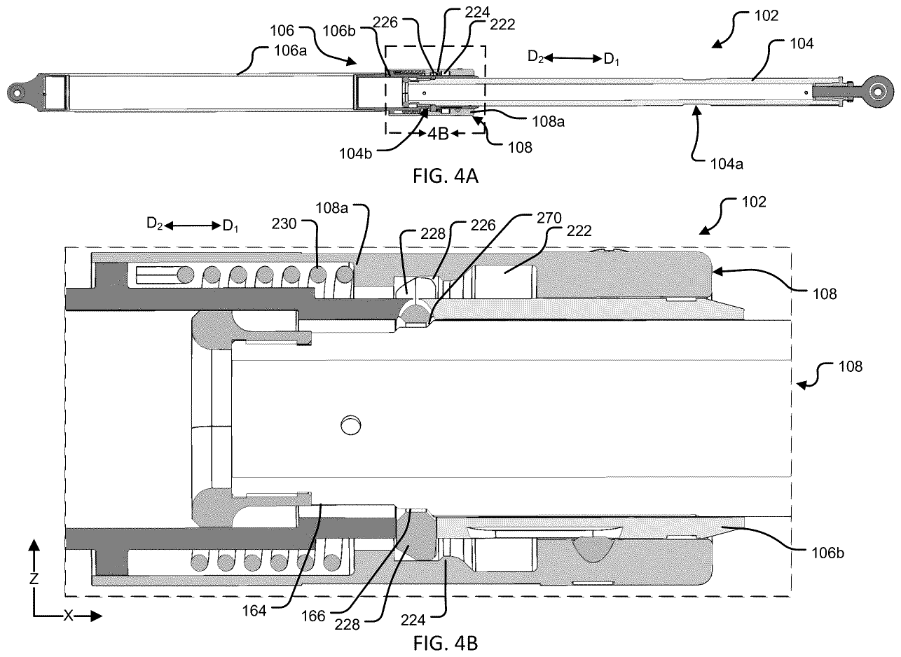

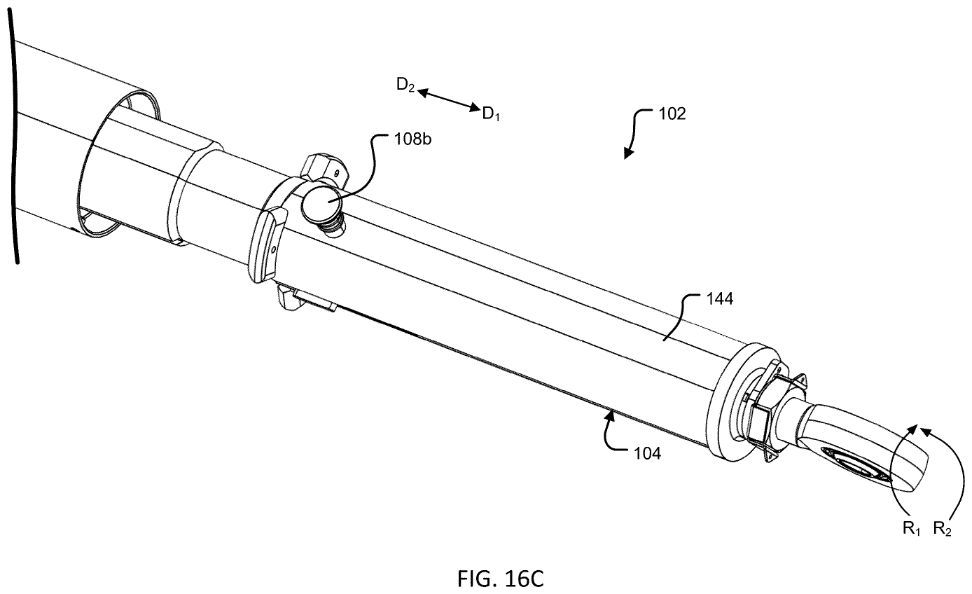

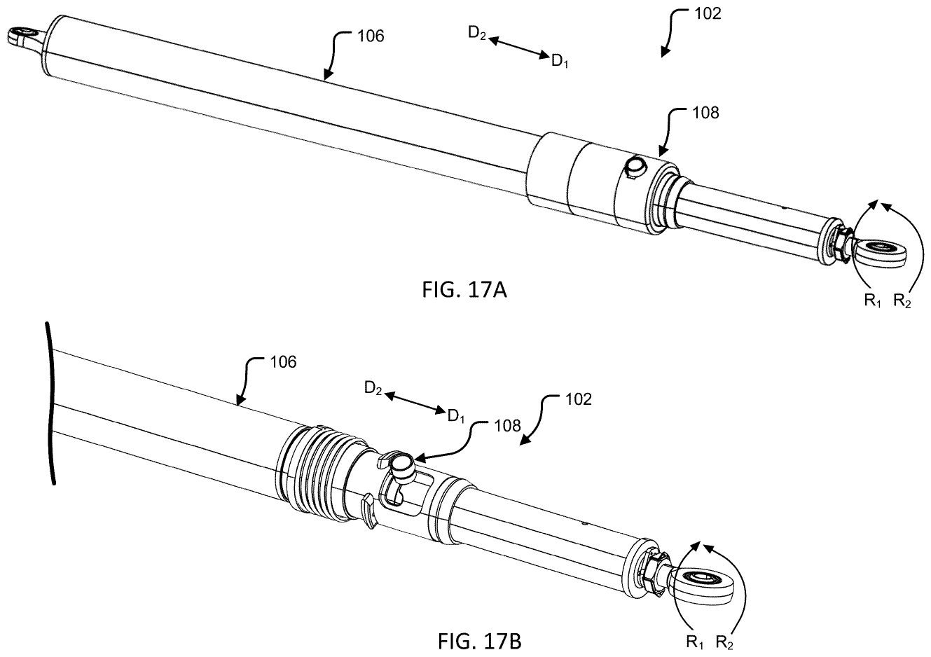

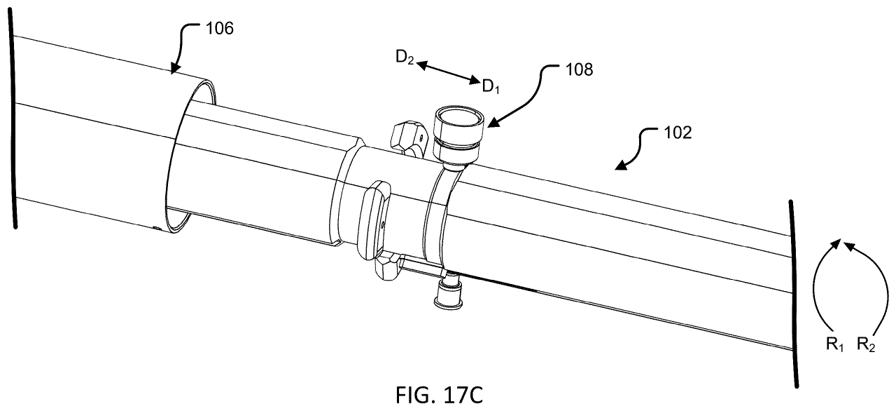

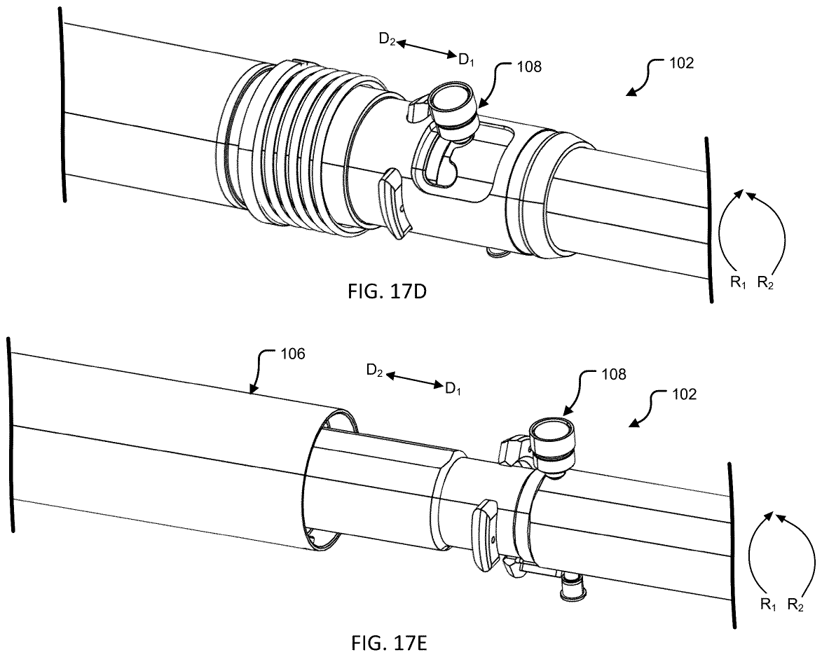

The inner tube 104 may include a first locking portion 104a and a second locking portion 104b that is offset from the first locking portion 104a in the second axial direction D2. When in an initial locked position (see e.g., FIGS. 2-6), the locking collar 108 is configured to lock the outer tube 106 at the second locking portion 104b of the inner tube 104. The locking collar 108 may be configured such that the locking collar 108 prevents the outer tube 106 from translating relative to the inner tube 104 in the first axial direction D1 beyond an initial position (e.g., the extended configuration of the hold open rod 102 in FIG. 4) that is offset from a predetermined position in the second direction D2 (see e.g., FIGS. 17A-17E).

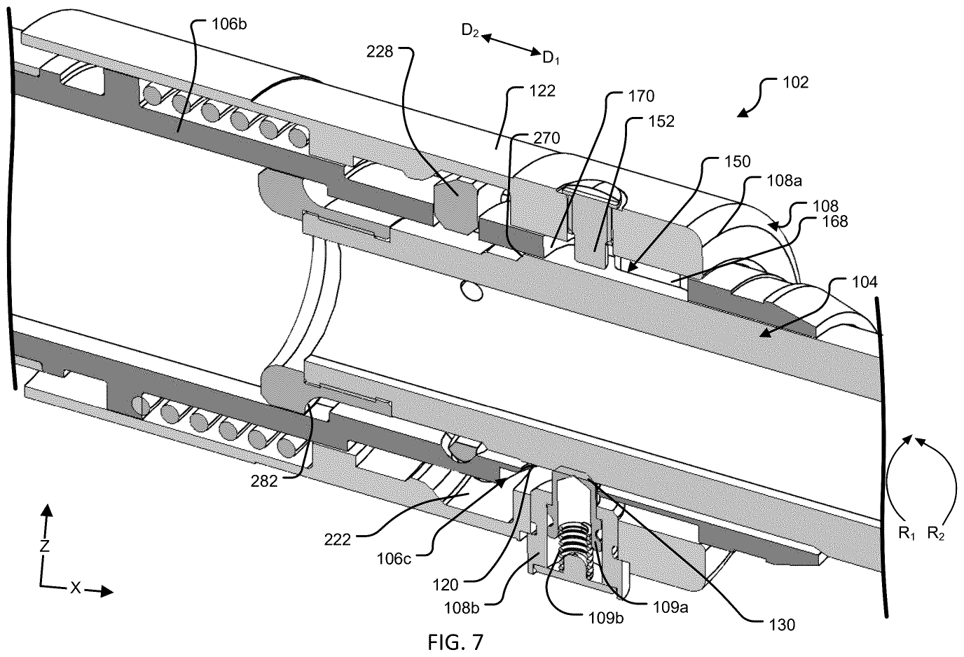

The locking collar 108 may be movable to a first unlocked position (see e.g., FIGS. 7-8) and a second unlocked position (see e.g., FIGS. 17A-17C). The locking collar 108 may be configured to move (e.g., rotate) between the first unlocked position and the second unlocked position, as discussed further below.

Movement of the locking collar 108 between the first unlocked position and the second unlocked position may transition the hold open rod 102 between a first mode and a second mode. The hold open rod 102 may be configured to be in the first mode when the locking collar 108 is in the first unlocked position. For example, when in the first mode, the locking collar 108 may be configured to allow the outer tube 106 to move in the first axial direction D1 relative to the inner tube 104 beyond the initial position.

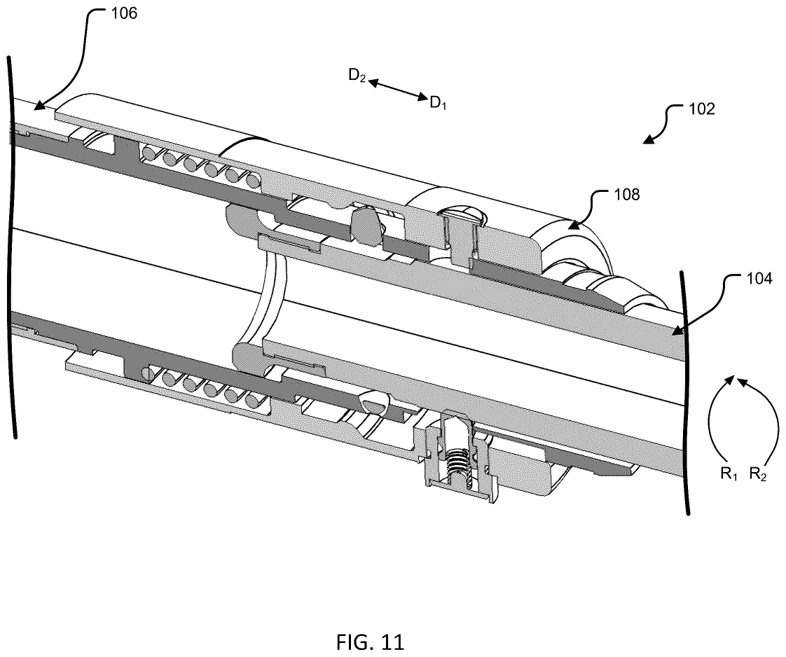

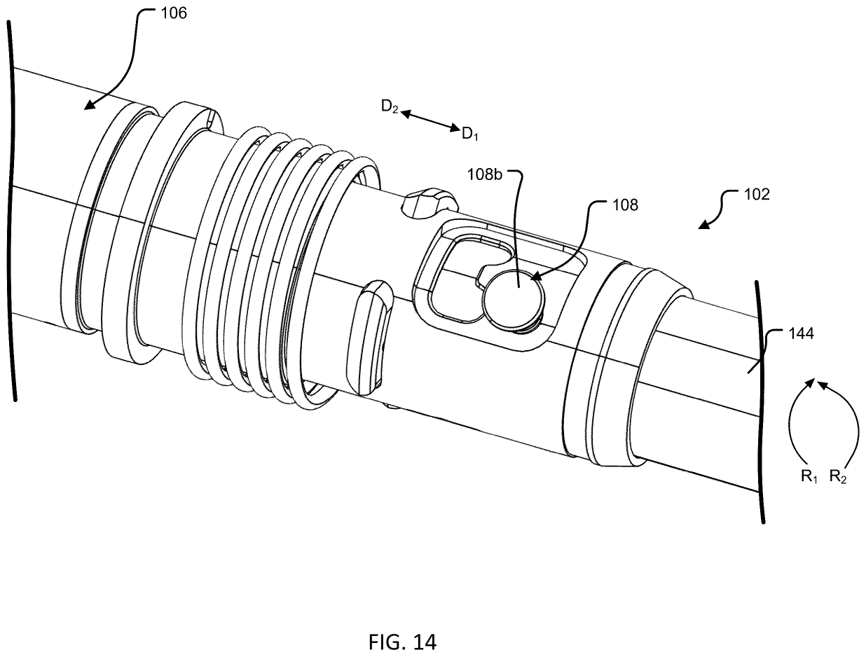

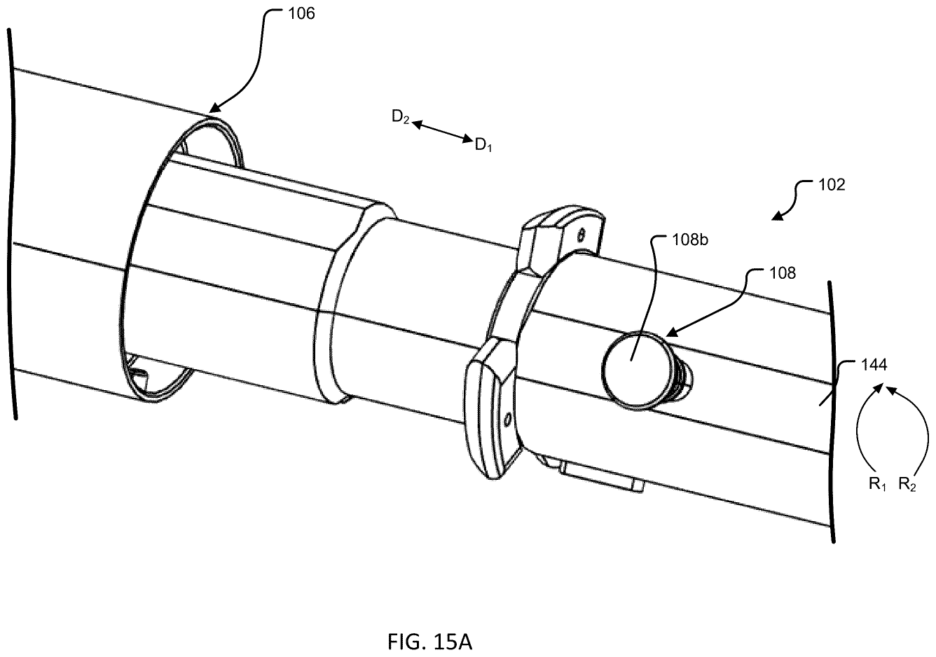

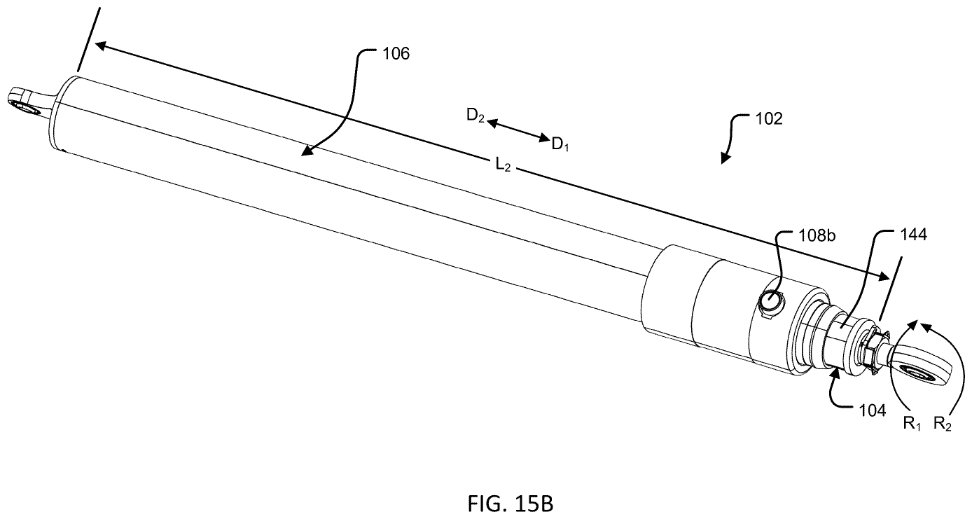

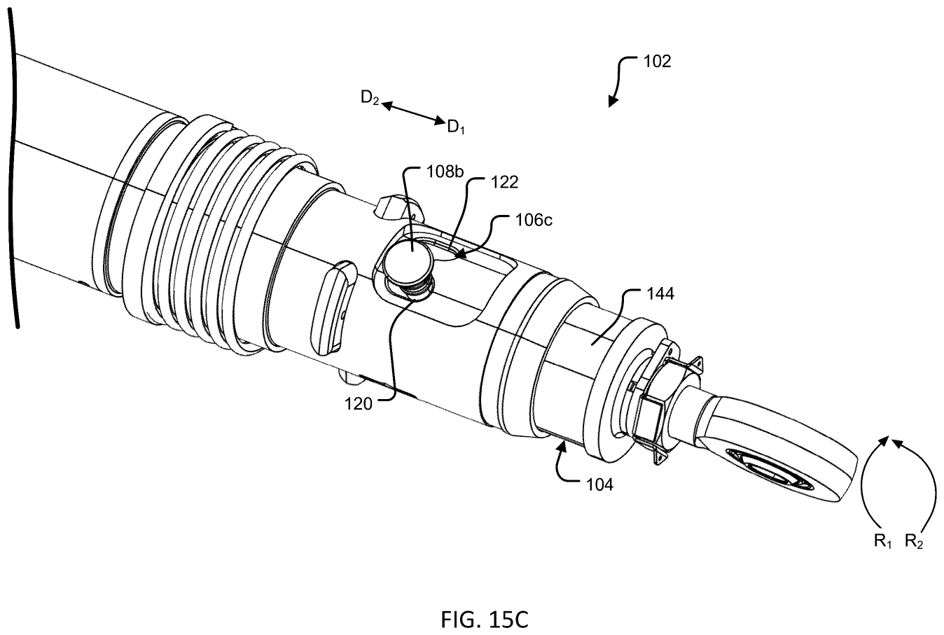

Referring now to FIGS. 8-11, when the hold open rod 102 is in the first mode, the locking collar 108 may be configured to lock the outer tube 106 and the inner tube 104 when the outer tube 106 translates relative to the inner tube 104 in the first axial direction D1 such that the locking collar 108 moves to the first locking portion 104a of the inner tube 104 and prevents the outer tube 106 from translating relative to the inner tube 104 in the first axial direction D1 beyond the predetermined position (see e.g., FIGS. 14-15A). For example, the locking collar 108 may prevent outer tube 106 and the inner tube 104 from compressing together beyond the intermediately compressed configuration (e.g., to a fully compressed configuration or a stowed configuration).

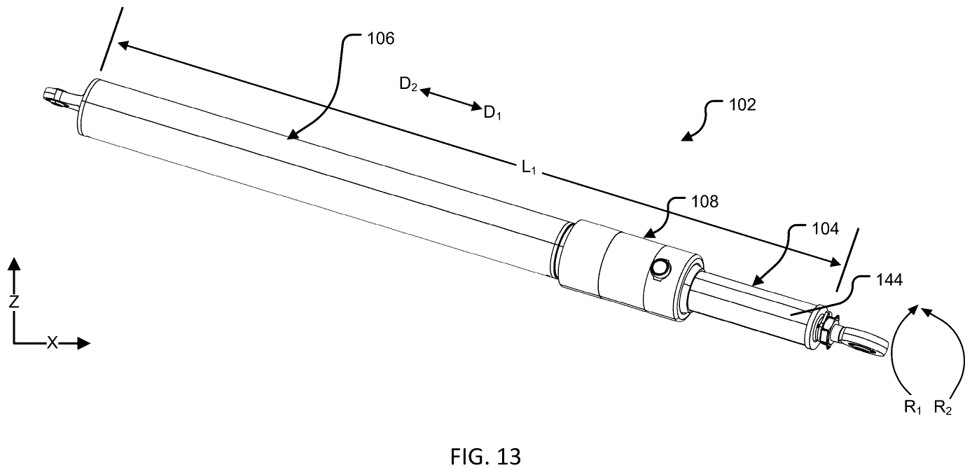

When in the first mode the locking collar 108 may be configured to allow the outer tube 106 and the inner tube 104 to compress to a first compressed configuration (e.g., FIGS. 13-15A) and prevent the outer tube 106 and the inner tube 104 from compressing beyond the first compressed configuration, in which the outer tube and the inner tube 104 together have a first length L1.

The locking collar 108 may be configured to engage the first locking portion 104a such that the outer tube 106 and the inner tube 104 are locked when the outer tube reaches the predetermined position. Thus, the locking collar 108 may prevent compression of the hold open rod 102 when the locking collar 108 is engaged with the first locking portion. In an embodiment, the locking collar may prevent extension and compression of the hold open rod when the locking collar is engaged with the first locking portion. In another embodiment, the locking collar may prevent extension but not compression of the hold open rod when the locking collar is engaged with the first locking portion.

When locked, the locking collar 108 may be configured to prevent further compression of the outer tube 106 and the inner tube 104, but allow for at least some extension of the outer tube 106 and the inner tube 104. In an embodiment, the locking collar is configured to not allow any extension of the outer tube and the inner tube when locked.

The hold open rod 102 may be configured to be in the second mode when the locking collar 108 is in the second unlocked position. For example, when in the second mode, the locking collar 108 may be configured to not lock the outer tube 106 and the inner tube 104 when the outer tube 106 translates relative to the inner tube 104 in the first axial direction D1 to the first locking portion 104a of the inner tube 104 such that the locking collar 108 allows the outer tube 106 to translate relative to the inner tube 104 to and beyond the predetermined position (see e.g., FIGS. 14-15A).

When in the second mode, the locking collar 108 may be configured to allow the outer tube 106 and the inner tube 104 to compress to the first compressed configuration (e.g., FIGS. 13-15A) and compress beyond the first compressed configuration, in which the outer tube and the inner tube 104 together have a second length L2 (e.g., FIG. 15B) that is less than the first length L1.

When in the second mode, the locking collar 108 may be configured to allow the outer tube 106 to move in the first axial direction D1 relative to the inner tube 104 beyond the initial position (e.g., FIG. 2). The locking collar 108 may be configured to not engage the first locking portion 104a when the outer tube 106 reaches the predetermined position. Thus, the outer tube 106 and the inner tube 104 may remain unlocked, thereby allowing the outer tube 106 to move in the first axial direction relative to the inner tube 104 beyond the predetermined position (e.g., FIGS. 13-15A) to a more compressed configuration (see e.g., FIG. 15B).

The locking collar 108 may allow the outer tube 106 and the inner tube 104 to compress together to the fully compressed configuration or the stowed configuration. Thus, the locking collar 108 may allow compression of the hold open rod 102 when the locking collar 108 is not engaged with the first locking portion. In an embodiment, the locking collar may allow extension and compression of the hold open rod when the locking collar is not engaged with the first locking portion. In another embodiment, the locking collar may allow extension but not compression of the hold open rod when the locking collar is not engaged with the first locking portion.

As discussed above, reference to movement of the outer tube 106 relative to the inner tube 104 does not necessarily mean that the outer tube 106 moves in absolute three-dimensional space, but rather one or both of the outer tube 106 and the inner tube 104 may be moving in a manner that results in the described movement of the outer tube 106 relative to the inner tube 104, and vice versa. For example, translating the outer tube 106 relative to the inner tube 104 in the first axial direction D1 includes an example in which the outer tube 106 remains fixed to a stationary vehicle (e.g., mounted to the housing 204 in FIG. 1 while the housing 204 is stationary) while the inner tube 104 translates in the second axial direction D2 with the door component 202 (e.g., the cowling).]

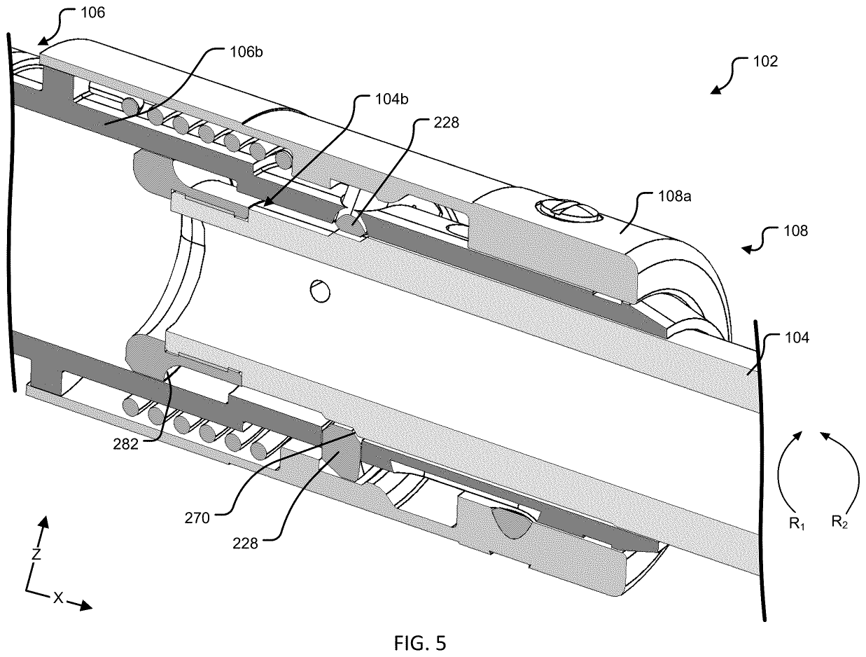

Turning to FIGS. 5-10A, the locking collar 108 may be rotatably movable between the first unlocked position and the second unlocked position. For example, the locking collar 108 may include a locking collar body 108a and a locking pin 108b that is coupled to the locking collar body 108a. The locking collar body 108a may be configured to rotate in a first rotational direction (e.g., clockwise when looking toward the locking collar 108 in the second direction D2 from an outer end of the inner tube 104) about the longitudinal axis X such that the locking pin 108b travels with the locking collar body 108a.

For example, the second elongated tubular body 106b may include an unlocking channel 106c that is configured to receive the locking pin 108b. The unlocking channel 106c may face radially outwardly. The unlocking channel 106c may be disposed at or near an inner end of the second elongated tubular body 106b. In some embodiments, the unlocking channel is formed at another suitable location of the outer tube, such as at an inner end of the first elongated body.

The unlocking channel 106c may include a first portion 120 and a second portion 122 that is offset from the first portion 120. For example, the second portion 122 of the unlocking channel 106c may be circumferentially offset from the first portion 120 along the first rotational direction R1.

The unlocking channel 106c may be U-shaped or bean shaped (e.g., kidney shaped). In another embodiment, the unlocking channel has a different shape that is configured to retain the locking pin in at least two different positions while pinned in the unlocking channel.

The locking pin 108b may be configured to contact a radially outwardly facing recessed surface 106d of the outer tube 106 when the locking collar 108 is in the locked position. For example, the locking pin 108b may be configured to remain outside of the unlocking channel 106c and in contact with the radially outwardly facing recessed surface 106d when the locking pin 108b is unpinned from the unlocking channel 106c.

The locking pin 108b may include a movable member 109a and a pin biasing member 109b (see e.g., FIG. 7). The pin biasing member 109b may be configured to urge the movable member 109a radially inwardly.

When the locking collar 108 is unlocked, the locking pin 108b may be disposed within the unlocking channel 106c. The locking pin 108b may be configured to contact an interior surface 124 that defines the unlocking channel 106c.

The locking pin 108b may be configured to move radially outwardly to reach the radially outwardly facing recessed surface 106d when the locking pin 108b is in a partially radially outward position and urged against the interior surface 124. For example, the interior surface 124 may be contiguous with the radially outwardly facing recessed surface 106d, and the interior surface 124 may be chamfered. The chamfering or other suitable feature of the interior surface 124 may be configured to move the locking pin 108b to the partially radially outward position when the locking pin 108b contacts a first curved outer surface 126 of the inner tube 104 and is urged against the interior surface 124.

A radially inward end 130 (see e.g., FIG. 7) of the locking pin 108b (e.g., the movable member 109a) may be configured to urge the locking pin 108b radially outwardly to the partially radially outward position. For example, the radially inward end 130 may be chamfered or include another suitable feature that is configured to move the locking pin 108b radially outwardly out of the unlocking channel 106c to the partially radially outward position. The movable member 109a may be configured to move radially outwardly upon being moved against the inner tube 104 and/or the outer tube 106. For example, the movable member 109a may be chamfered at its radially inward end such that the movable member 109a retracts radially outwardly upon contacting the interior surface 124 that defines the unlocking channel 106c.

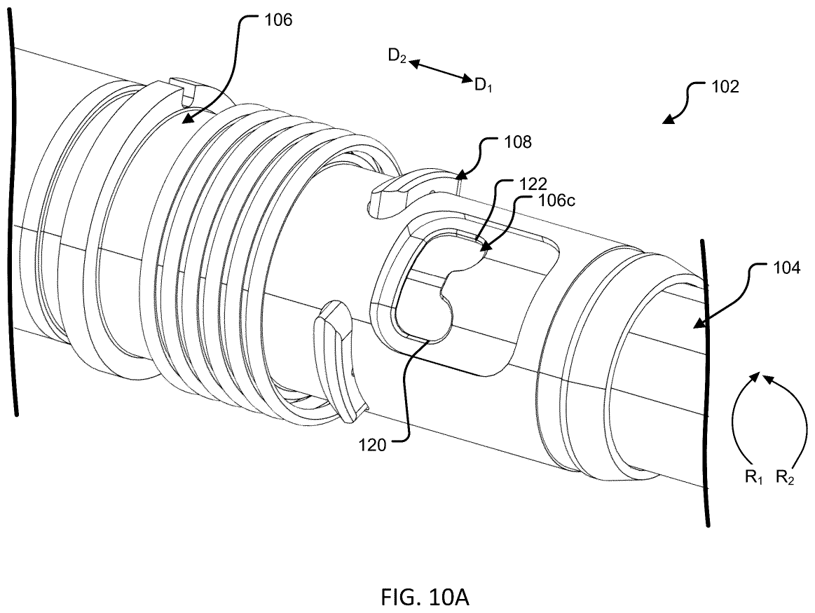

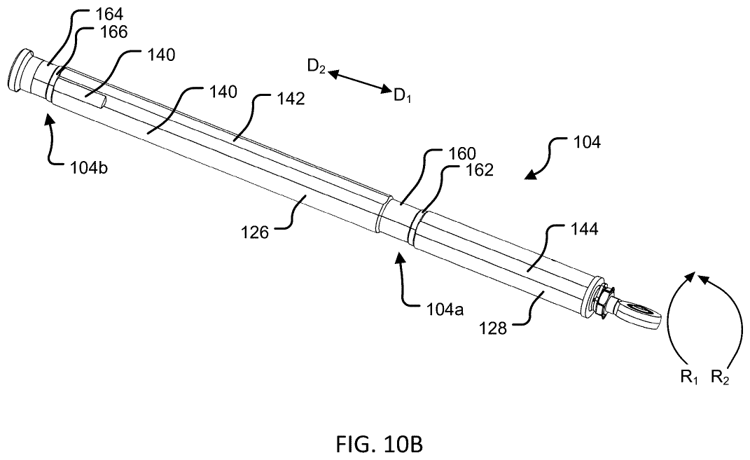

The locking pin 108b may be configured to follow along a first path when in the first mode and may be configured to follow along a second path when in the second mode. For example, with brief reference to FIG. 10B, the inner tube 104 may include a first radially recessed portion 140 that extends in the first direction D1 to the first curved outer surface 126, which may be radially outward of the first radially recessed portion 140. The first radially recessed portion 140 may thus extend only partially from the second locking portion 104b in the first axial direction D1 toward the first locking portion 104a.

The first curved outer surface 126 may be cylindrical. In an embodiment, the first curved outer surface has another suitable shape that has a radially outward extent greater than the second recessed portion, at least along the pathway that the locking pin follows when in the first mode. For example, the inner tube may include a protrusion in the pathway of the locking pin that causes the locking pin to unpin from the unlocking channel.

The inner tube 104 may include a second radially recessed portion 142 that extends in the first direction D1 from the second locking portion 104b to the first locking portion 104a. The second radially recessed portion 142 may be circumferentially offset from the first radially recessed portion (e.g., in the first rotational direction R1).

The first locking portion 104a may include a primary radially recessed surface 160 (which can be referred to as a reset surface) and an intermediately radially recessed surface 162 (which can be referred to as a locking surface) that are each radially recessed from the first curved outer surface 126. The intermediately radially recessed surface 162 may be disposed radially outwardly of the primary radially recessed surface 160.

The inner tube 104 may include a third radially recessed portion 144, that extends in the first axial direction D1 from the first locking portion 104a to an outer end of the inner tube 104. The third radially recessed portion 144 may be axially offset from the first radially recessed portion 140 and the second radially recessed portion 142 in the first axial direction D1. The third radially recessed portion 144 will be discussed further below with respect to extension the hold open rod 102 from the compressed configuration.

When in the second mode, the locking collar 108 may be configured to transition to a transitional unlock position (see e.g., FIG. 17D) when the locking collar 108 moves past the first locking portion 104a in the first axial direction D1. For example, the locking pin 108b may be configured to unpin from the unlocking channel 106c (e.g., unpin from the second portion 122) as the locking pin 108b translates over a second curved outer surface 128 of the inner tube 104. Accordingly, the locking collar 108 may be in the transitional unlock position when the locking pin 108b is unpinned.

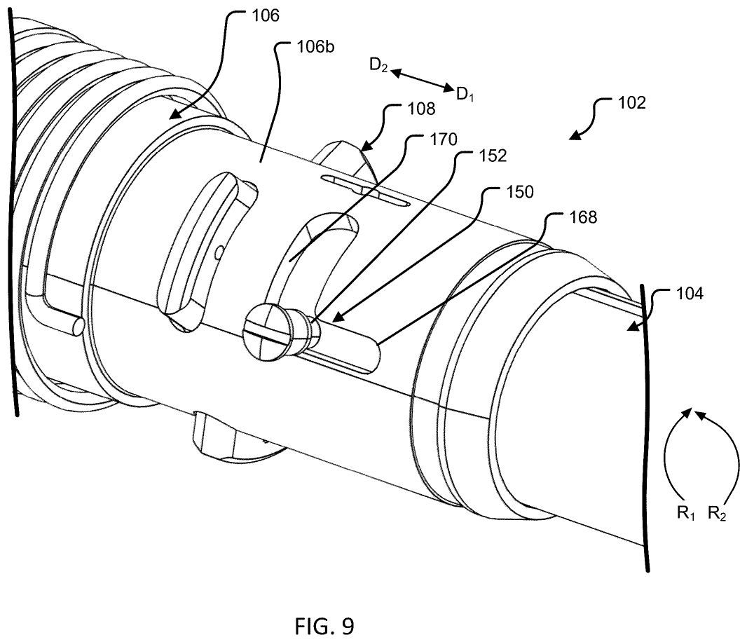

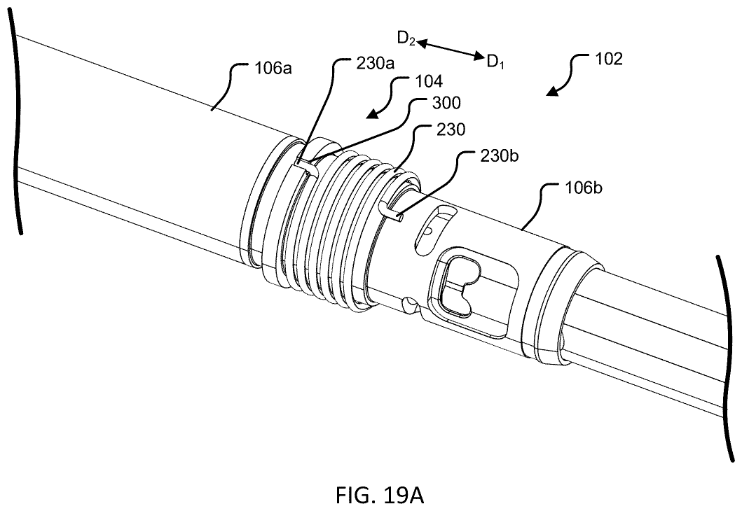

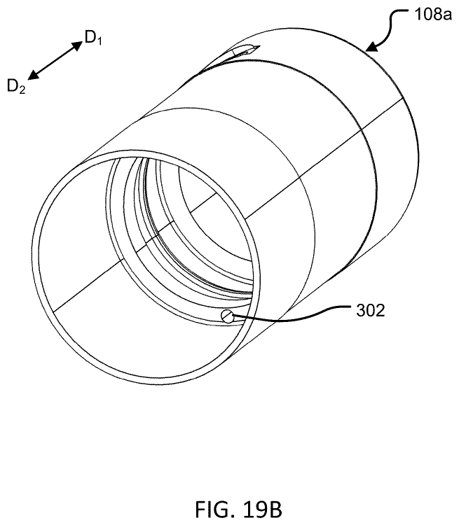

While the hold open rod 102 is in the second mode and the locking collar 108 is in the transitional unlock position, the locking collar 108 may be configured to transition to the first unlocked position, as discussed further below with respect to FIGS. 9 and 19A-19B. In another embodiment, when the locking collar is in the transitional unlock position, the locking collar may be configured to transition to the locked position by moving the locking collar over another locking portion in the second axial direction D2. For example, when such a hold open rod is compressed to a length less than its intermediate length (e.g., compressed to its minimum length) while the locking collar is in the transitional unlock position, the locking collar may be configured to lock the hold open rod.

When the hold open rod 102 is extended back to its intermediate length (i.e., intermediately extended position), the locking collar 108 may be configured to remain unlocked upon translating to the first locking portion 104a in the second direction D2. This process is discussed in more detail further below with respect to FIGS. 5-10 and 17A-18B. In other embodiments, the locking collar may be configured to remain pinned in the second unlocked and pinned position after moving past the first locking portion in the first direction.

The second curved outer surface 128 of the inner tube 104 may be disposed in the first axial direction D1 from the first curved outer surface 126. For example, the first locking portion 104a may extend between the first curved outer surface 126 and the second curved outer surface 128. The second curved outer surface 128 may be cylindrical. In an embodiment, the second curved outer surface has another suitable shape that has a radially outward extent greater than the second recessed portion, at least along the pathway that the locking pin follows when in the second mode. For example, the inner tube may include a protrusion in the pathway of the locking pin that causes the locking pin to unpin from the unlocking channel.

The primary radially recessed surface 160 and the intermediately radially recessed surface 162 may each extend circumferentially such that the primary radially recessed surface 160 and the intermediately radially recessed surface 162 define at least a portion of a circumferentially extending groove between the first curved outer surface 126 and the second curved outer surface 128.

The second locking portion 104b may include a primary radially recessed surface 164 and an intermediately radially recessed surface 166 that are each radially recessed from the first curved outer surface 126. The intermediately radially recessed surface 166 may be disposed radially outwardly of the primary radially recessed surface 164.

The primary radially recessed surface 164 and the intermediately radially recessed surface 166 may each extend circumferentially such that the primary radially recessed surface 164 and the intermediately radially recessed surface 166 define at least a portion of a circumferentially extending groove that extends in the second axial direction D2 away from the first curved outer surface 126.

When the hold open rod 102 is in the first mode, the locking pin 108b is configured to translate with the outer tube 106 along the first radially recessed portion 140 of the inner tube 104 to the first curved outer surface 126. The first curved outer surface 126 of the inner tube 104 that is configured to urge the locking pin 108b radially outward into an unpinned position in which the locking pin 108b is outside of the unlocking channel 106c. When the locking pin 108b is unpinned by the first curved outer surface 126 while the hold open rod 102 is in the first mode, the locking collar 108 may transition from the first unlocked position to a first transitional unlocked and unpinned position. In some embodiments, another protrusion or inclined surface is configured to urge the locking pin radially outward at an end of the first radially recessed portion.

The hold open rod 102 may be in the first mode when the locking collar 108 is in the first unlocked position and when in the locking collar 108 is in the first transitional unlocked and unpinned position. For example, the hold open rod 102 may remain in the first mode as the locking collar 108 transitions through positions while the hold open rod 102 is compressed, until the locking collar 108 locks at the first locking portion 104a.

Turning to FIGS. 7-9, the second elongated tubular body 106b may include a guiding channel 150 that is configured to receive a guiding pin 152 (an example of a guide member) that is fixed relative to the locking collar body 108a. For example, guiding channel 150 may open radially outwardly (e.g., may define a through hole in the second elongated tubular body 106b). The guiding channel 150 may be disposed opposite the unlocking channel 106c, at or near the inner end of the second elongated tubular body 106b. In some embodiments, the guiding channel is formed at another suitable location of the outer tube, such as at an inner end of the first elongated body.

The guiding channel 150 may include a first guide portion 168 and a second guide portion 170 that is at least partially offset from the first guide portion 168. For example, the first guide portion 168 of the guiding channel 150 may extend axially (e.g., from an end of the second guide portion 170, and axially beyond the entirety of the second guide portion 170 in the first axial direction D1). The second guide portion 170 of the guiding channel 150 may extend circumferentially (e.g., from an end of the first guide portion 168, and circumferentially beyond the entirety of the first guide portion 168 in the first rotational direction R1).

The guiding channel 150 may be L-shaped or another suitable shape that is configured to guide the locking collar body 108a while transitioning the locking collar 108 to each unlocked position while the locking pin 108b is pinned and/or back to the locked position while the locking pin 108b is unpinned. In another embodiment, the guiding channel has a different suitable shape that is configured to guide the guiding pin, and thus the locking collar body while the locking collar transitions to each unlocked position while the locking pin is pinned and/or back to the locked position while the locking pin is unpinned.

The guiding pin 152 may be configured to remain within the guiding channel 150 regardless of whether the locking collar 108 is locked or unlocked. For example, the guiding pin 152 may be configured to remain in the first guide portion 168 offset from the second guide portion 170 while the locking pin 108b is unpinned and the locking collar 108 is locked. The guiding pin 152 may be configured to remain in the second guide portion 170 offset from the first guide portion 168 while the locking pin 108b is pinned and the locking collar 108 is unlocked.

Thus, the guiding pin 152 may be configured to simultaneously move within the guiding channel 150 each time the locking pin 108b moves circumferentially or axially relative to the second elongated tubular body 106b.

The guiding pin 152 may be a screw that is threadedly coupled to the locking collar body 108a and protrudes radially inwardly into the guiding channel 150. In some embodiments, the guiding pin is a radially inward protrusion of the locking collar body or another suitable feature that is configured to be received in the guiding channel. In another embodiment, the locking collar body includes a radially inwardly opening guiding channel and the second elongated body or the first elongated body includes a radially outwardly extending protrusion that is received in the radially inwardly opening guiding channel.

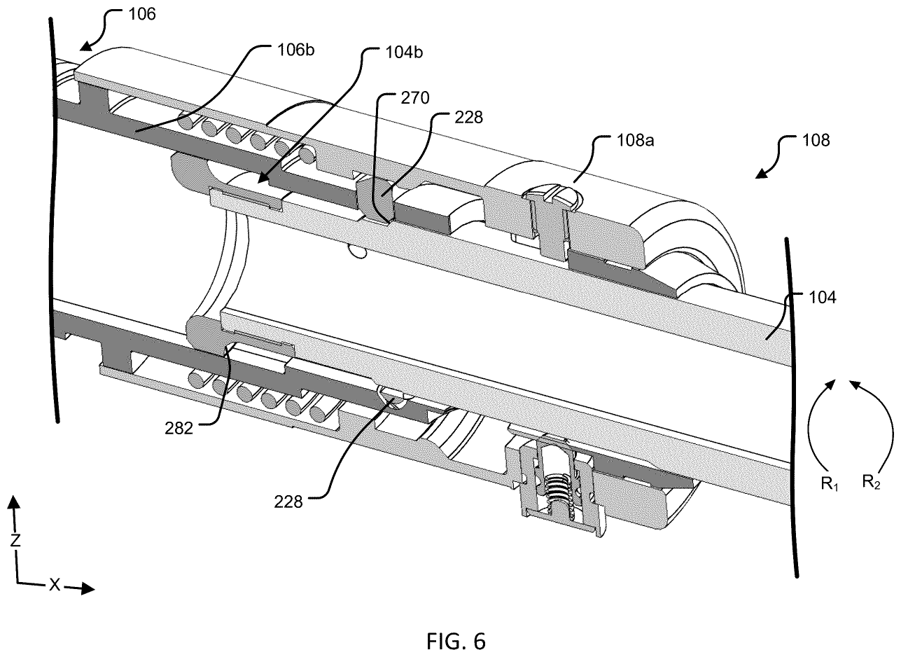

Turning to FIGS. 5-6, the locking collar 108 may be similar to the lock mechanism disclosed in U.S. Pat. No. 9,464,645 entitled “Pull then lift hold open rod lock mechanism” and issued on Oct. 11, 2016, which is hereby incorporated by reference in its entirety, except as noted herein.

As discussed above, the locking collar 108 may be configured to unlock the inner tube 104 and the outer tube 106, in which the inner tube 104 and the outer tube 106 are translatable relative to one another. When locked, the locking collar 108 may be configured to lock the inner the inner tube 104 and the outer tube 106 together, such that the inner tube 104 and the outer tube 106 are not translatable relative to one another (e.g., to compress and/or extend the hold open rod 102).

The locking collar 108 may include one or more locking dogs 228 (an example of one or more locking elements) that are configured to be received by the first locking portion 104a and the second locking portion 104b of the inner tube 104. For example, when the locking collar 108 is locked and the hold open rod 102 is in the extended configuration, the locking dogs 228 may be configured to abut an axially facing surface 270 that defines at least a portion of the second locking portion 104b. The locking collar 108 may be engaged with the second locking portion 104b when the locking collar 108 is in the locked position and the locking dogs 228 abut the axially facing surface 270 such that the locking collar 108 prevents the outer tube 106 from further translation in the first axial direction D1.

When the locking collar 108 is locked in the first compressed configuration (see e.g., FIGS. 13-15A, which may be referred to as an intermediately compressed configuration), locking dogs 228 may be configured to abut an axially facing secondary surface 284 (see e.g., FIGS. 18A-18B) that defines at least a portion of the first locking portion 104a. The locking collar 108 may be engaged with the first locking portion 104a (see e.g., FIG. 15A) when the locking collar 108 is in the locked position and the locking dogs 228 abut the axially facing surface 280 such that the locking collar 108 prevents the outer tube 106 from further translation in the first axial direction D1.

When the locking collar 108 is locked and locking dogs 228 abut the respective axially facing surface 270 or 280, the outer tube 106 and the inner tube 104 may be extendable. For example, the locking dogs 228 may be configured to move with the locking collar body 108a in the second axial direction D2 until reaching the corresponding secondary axially facing surface 282, 284. In an embodiment, the inner tube may include more than two locking portions that are configured to receive the locking dogs. For example, the inner tube may include three or more axially spaced apart groove that are configured to receive the one or more locking dogs. Each of the three or more axially spaced apart grooves may include a respective axially facing surface and/or opposing axially facing surface that is configured to abut the one or more locking dogs when the one or more dogs are received in the respective groove and the locking collar is locked.

Referring particularly to FIGS. 4A-4B, the locking collar body 108a may include an unlock groove 222 and a lock groove 226. The locking collar body 108a may include a no-unlock feature 224 arranged between the unlock groove 222 and lock groove 226.

The no-unlock feature 224 may be a radially inwardly extending protrusion. The no-unlock feature 224 may at least partially define the unlock groove 222 and the lock groove 226.

Each locking dog 228 may be configured to fit into and/or slide between the unlock groove 222 and lock groove 226. The no-unlock feature 224 may prevent the locking dog 228 from sliding between the unlock groove 222 and lock groove 226 when the hold open rod 102 is in the stowed configuration or a locked configuration.

When the hold open rod 102 is in the stowed configuration, each locking dog 228 may be configured to be disposed within the unlock groove 222. The unlock groove 222, the lock groove 226, the no-unlock feature 224, and each locking dog 228 may include chamfered, beveled, or curved radiused edges to assist in the movement of the locking dog 228 between the unlock groove 222 and the lock groove 226.

Each locking dog 228 may be formed from a linear element, such as a hexagonal prism, a segmented circle, a portion of a cylindrical ring, or cylinder, each with beveled edges. Each locking dog 228 may be made from a polyamide-imide material, such as Torlon®, or a metal, such as an aluminum alloy, a copper alloy, steel, or the like. Each locking dog 228 may be reinforced with glass fiber, carbon fiber, or the like. In an embodiment, the locking collar includes more than three locking dogs. In another embodiment, the locking collar includes one or two locking dogs.

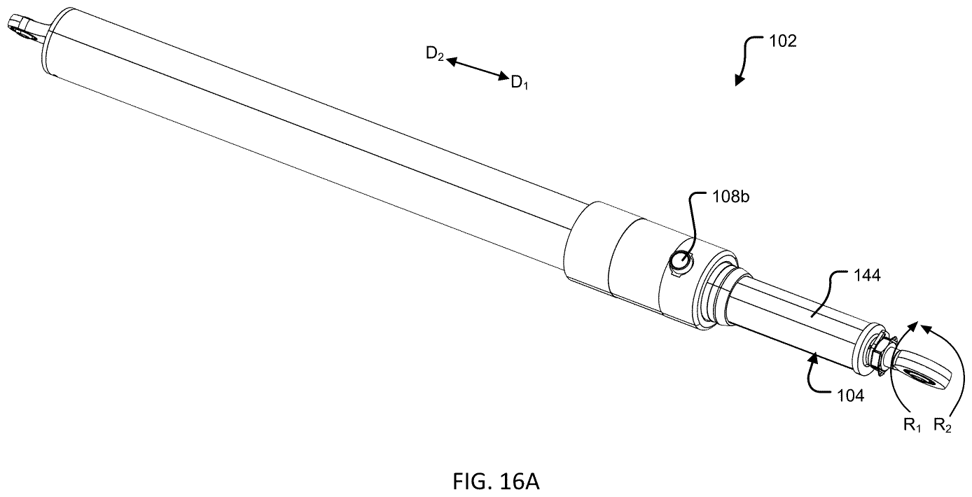

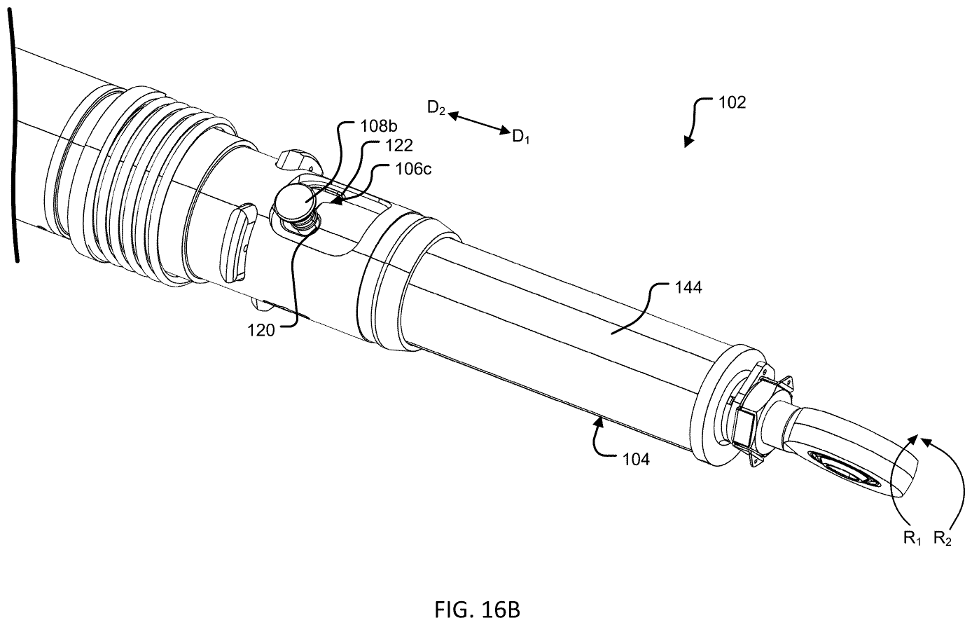

The hold open rod 102 may be locked and unlocked by moving the locking collar 108 to the locked position or one of the unlocked positions. For example, with reference to FIG. 10A, to lock the hold open rod 102 from the stowed configuration (see e.g., FIG. 16A), while the locking pin 108b is unpinned from the second portion 122 and pinned in the first portion 120 of the unlocking channel 106c, the inner tube 104 may be extended relative to the outer tube 106 in the first direction D1 until a desired length of the hold open rod 102 is reached. As the hold open rod 102 continues extending while the locking pin 108b remains pinned in the first portion 120 of the unlocking channel 106c, the inner tube 104 may move relative to the outer tube 106. For example, the hold open rod 102 may extend until the locking dogs 228 abut the opposing secondary axially facing surface 282 (see e.g., FIG. 5) of the inner tube 104. The locking pin 108b may be configured to unpin from the first portion 120 prior to the locking dogs 228 reaching the second locking portion 104b as the hold open rod 102 extends, as discussed below regarding unpinning of the locking pin 108b.

A biasing member 230 (e.g., a coil spring) of the hold open rod 102 may be configured to urge the locking collar 108 (e.g., the locking collar body 108a) in the first direction D1. For example, when the locking dogs 228 translate to the primary radially recessed surface 164 (or the primary radially recessed surface 164 shown in FIG. 10B) of the respective first locking portion 104a (or the second locking portion 104b shown in FIG. 10B), the biasing member 230 may urge the locking collar body 108a to move in the first direction D1 relative to the locking dogs 228.

While the locking pin 108b is pinned in the unlocking channel 106c, the locking pin 108b may be configured to prevent the biasing member 230 from causing the locking collar body 108a to move in the first direction D1 relative to the locking dogs 228. When the locking pin 108b is unpinned from the unlocking channel 106c, the biasing member 230 may be configured to cause the locking collar body 108a to move in the first direction D1 relative to the locking dogs 228.

When the locking pin 108b is unpinned from the unlocking channel 106c, as the locking collar body 108a moves in the first direction D1 relative to the locking dogs 228, the locking collar body 108a may be configured to move the locking dogs 228 radially inwardly to the respective primary radially recessed surface 160 or 164 so that the no-unlock feature 224 may move axially relative to the locking dogs 228 in the first axial direction D1 from its position illustrated in FIG. 7. The locking collar body 108a may be configured to move relative to the locking dogs 228 in the first axial direction D1 such that the locking dogs 228 are received in the lock groove 226 and the locking dogs 228 engage with the corresponding one of the first locking portion 104a or the second locking portion 104b (see e.g., FIG. 4B or FIG. 15A, which illustrates an oblique view of the locking dogs 228 engaged with the first locking portion 104a of the inner tube 104).

When the hold open rod 102 is in the stowed configuration (see e.g., FIG. 16A), and the biasing member 230 may be configured to urge the locking collar 108 in the first axial direction D1 away from the axially facing surface 270 (FIG. 6) of the outer tube 106. While the locking pin 108b remains pinned in the first portion 120, locking collar body 108a may be prevented from moving in the first axial direction D1 relative to the locking dogs 228 such that the locking dogs 228 are received in the lock groove 226. Thus, the locking collar 108 may be configured to remain unlocked, until the locking pin 108b is unpinned from the unlocking channel 106c.

The locking pin 108b may be configured to unpin from the unlocking channel 106c (e.g., from the first portion 120 of the unlocking channel 106c) when the locking pin 108b is translated relative to the inner tube 104 in the second axial direction D2 to the first curved outer surface 126. The locking pin 108b may be configured to unpin from the unlocking channel 106c (e.g., from the first portion 120 of the unlocking channel 106c) upon reaching the first curved outer surface 126. The locking dogs 228 may be configured to be offset in the second direction D2 from the first locking portion 104a, upon pinning of the locking pin 108b, such that the locking dogs 228 are unable to move radially inward of the first curved outer surface 126 (and/or the second curved outer surface 128) of the inner tube 104.

The primary radially recessed surfaces 160 and 164 may define respective reset grooves that are deeper than the respective intermediately radially recessed surface 162 or 166, to allow the locking dogs 228 to move into the lock groove 226. The biasing member 230 may be configured to move the no-unlock feature 224 in the first axial direction D1 past the locking dogs 228 so that the locking dogs 228 are disposed within the lock groove 226. The locking dogs 228 may be configured to remain in the lock groove 226, until the locking collar body 108a is moved (e.g., by an operator) in the second axial direction D2 relative to the outer tube 106 (e.g., while the locking dogs are in one of the reset grooves).

The biasing member 230 may be configured to circumferentially bias the locking collar 108 into the first unlocked position. For example, the biasing member 230 may be configured to bias the locking collar body 108a in a second rotational direction R2 (see e.g., FIG. 5) that is opposite the first rotational direction R1 relative to the unlocking channel 106c such that the locking pin 108b is urged circumferentially in the second rotational direction R1 toward the second portion 122 of the unlocking channel 106c.

One end of the biasing member may be circumferentially fixed to the outer tube 106. For example, the second elongated tubular body 106b may include a radially outwardly extending locking notch 300 that is configured to receive an outer end 230a (see e.g., FIG. 19A) of the biasing member 230 and prevent relative circumferential movement therebetween.

An inner end 230b of the biasing member 230 may be circumferentially fixed to the locking collar body 108a. For example, the locking collar body 108a may include a biasing member channel 302 (see e.g., FIG. 19B) that is configured to receive the inner end 230b of the biasing member 230 and prevent relative circumferential movement therebetween.

In an alternative embodiment, the biasing member is configured to circumferentially bias the locking collar into the second unlocked position. In some embodiments, the biasing member is configured to bias the locking collar in a different direction to urge the locking collar into the first unlocked position or the second unlocked position. For example, in an alternative embodiment, the biasing member may be configured to axially bias the locking collar into the first unlocked position or the second unlocked position.

Compression of the hold open rod 102 until the locking dogs 228 contact the respective intermediately radially recessed surface 162 or 166 may cause the locking dogs 228 to be secured in the lock groove 226, until the hold open rod 102 is extended again such that the locking dogs 228 are axially offset from the respective intermediately radially recessed surface 162 or 166 and thus able to move radially inwardly.

Turning to FIGS. 15B-16C, a transition of the hold open rod 102 from the compressed configuration, in which the locking pin 108b may be pinned, to the intermedi ately extended configuration is shown. The locking pin 108b may be configured to follow along a third path when the locking collar 108 is in the first unlocked position and the hold open rod 102 is expanded from the compressed configuration. For example, the locking pin 108b may be configured to translate along the third radially recessed portion 144 in the second axial direction D2.

The third radially recessed portion 144 may be axially aligned with the first radially recessed portion 140 (see e.g., FIG. 10B). Thus, the third path may follow along the first path, except in the opposite direction (i.e., in the second direction D2) relative to the inner tube 104. For example, the locking pin 108b may be configured to contact and/or translate along the third radially recessed portion 144 when pinned in the first portion 120 (an example of a primary pinned position) of the unlocking channel 106c.

The locking collar 108 may be configured to translate along the inner tube 104 so that the hold open rod 102 is able to expand from its fully compressed configuration (or intermediately compressed configuration) to its fully extended configuration, once the locking collar 108 is in the first unlocked position. For example, the locking pin 108b may be configured to remain pinned in the first portion 120 of the unlocking channel 106c, until the hold open rod 102 extends such that the locking pin 108b reaches the first curved outer surface 126, thereby causing the locking pin 108b to unpin.

When the locking pin 108b is unpinned from the second portion 122, the locking collar 108 may be configured to remain unlocked such that the hold open rod 102 may fully extend. For example, the locking dogs 228 (see e.g., FIG. 7) may be configured to remain in the unlock groove 222 such that the locking dogs 228 are movable radially outward of the first curved outer surface 126 as the locking collar 108 translates past the first locking portion 104a in the second axial direction D2. The locking collar 108 may be configured to transition to the first unlocked position after transitioning to the transitional unlocked position from the second unlocked position.

Accordingly, if the hold open rod 102 is extended to its fully extended position, the locking collar 108 may be configured to lock the hold open rod 102 in the second locking portion 104b at the fully extended position (or slightly compressed therefrom as discussed above to account for the locking dogs 228 entering the reset groove prior to locking at the intermediately radially recessed surface 166). Alternatively, if the hold open rod 102 is compressed to its intermediately extended position, the locking collar 108 may be configured to lock the hold open rod 102 in the first locking portion 104a. In some embodiments, alternative or additional paths (e.g., recessed portions in the inner tube) may be utilized to provide additional or additional options for locking or unlocking the hold open rod in different configurations as the hold open rod extends or compresses.

With reference to FIGS. 5-10B and 12-18B use of the hold open rod 102 will be discussed. When the hold open rod 102 is in the expanded configuration, the locking collar 108 may be locked such that the hold open rod 102 cannot compress to the intermediate configuration or the compressed configuration. For example, the locking pin 108b may be unpinned and the locking dogs 228 may interfere with movement of the outer tube 106 in the first axial direction relative to the inner tube 104. The locking dogs 228 may interfere with such movement by being retained in the lock groove 226 in contact with the axially facing surface 270, the intermediately recessed radially surface 166, and the no-unlock feature 224.

The door component 202 (FIG. 1) may be held open by the hold open rod 102 when the hold open rod 102 is locked in the extended position.

While the hold open rod 102 is in the extended position, the operator may transition the hold open rod 102 to the first mode or the second mode to unlock the locking collar 108 and allow the hold open rod 102 to compress. For example, the operator may transition the hold open rod 102 to the first mode by moving the locking collar 108 to its first unlocked position (e.g., by moving the locking collar body 108a in the second axial direction D2 relative to the outer tube 106 until the locking pin 108b is received in the first portion of the unlocking channel 106c). Moving the locking collar 108 to its first unlocked position may include partially extending the hold open rod 102 (e.g., anywhere from 1% to 10% of the fully extended length of the hold open rod 102) so that the locking dogs 228 move in the second axial direction D2 relative to the intermediately radially recessed surface 166 to reach the reset groove defined by the primary radially recessed surface 164.

Once the locking dogs 228 reach the reset groove, the locking collar body 108a may be moved (e.g., by the operator) in the second axial direction D2 relative to the outer tube 106 such that the no-unlock feature 224 translates past the locking dogs 228 until the locking dogs 228 are disposed in the unlock groove 222. Thus, the locking collar 108 may be in its first unlocked position, and as a result the hold open rod 102 may be in the first mode.

From the first unlocked position, the locking collar 108 may be rotated in the first rotational direction R1 to its second unlocked position. For example, the locking collar body 108a may be rotated in the first rotational direction R1 relative to the outer tube 106 until the locking pin 108b is received in the second portion 122 (an example of a bypass pinned position) of the unlocking channel 106c. Rotating the locking collar body 108a in the first rotational direction R1 may cause the locking pin 108b, the guiding pin 152, and/or the inner end 230b of the biasing member 230 to simultaneously rotate in the first rotational direction R1 with the locking collar body 108a.

Once the locking pin 108b is received in the second portion 122 of the unlocking channel 106c, the locking collar 108 may be in its second unlocked position, and as a result the hold open rod 102 may be in the second mode.

In some embodiments, the locking collar transitions from its locked position to the second unlocked position without first reaching the first unlocked position. For example, the locking pin and the guiding pin may follow a different path (e.g., axially or rotationally) that does not lead through the first portion of the unlocking groove.

While in the first mode or the second mode, the hold open rod 102 may be unlocked. For example, the hold open rod 102 may be compressible and thus not support the wight of the door component 202 in its fully open position.

While in the first mode, compression of the hold open rod 102 to the intermediately extended configuration may lock the hold open rod 102. For example, while pinned in the first portion 120 of the unlocking channel 106c, the locking pin 108b may translate in the first axial direction D1 along the first path defined by the first radially recessed portion 140. As the locking pin 108b translates in the first axial direction D1 to the first curved outer surface 126, the first curved outer surface 126 may unpin the locking pin 108b resulting in the locking collar 108 transitioning to its first transitional unlocked and unpinned position, in which the locking dogs 228 are in the unlock groove 222.

When in the first transitional unlocked and unpinned position, the locking collar 108 may lock at the next locking portion (e.g., the first locking portion 104a) the locking collar 108 translates to. Thus, the hold open rod 102 becomes locked when compressed to its intermediately extended position, at which point the hold open rod 102 may support the weight of the door component 202 (FIG. 1) in the partially closed position.

For example, compressing the hold open rod 102 may include translating the locking dogs 228 in the first axial direction D1 to the reset groove defined by the primary radially recessed surface 160 of the first locking portion 104a. Once the locking dogs 228 are in the reset groove, the biasing member 230 may move the locking collar body 108a in the first axial direction D1 relative to the locking dogs 228 such that the no-unlock feature 224 moves relative to the locking dogs 228 until the locking dogs 228 are disposed in the lock groove 226. The movement of the locking collar body 108a may follow a path defined by the guiding channel 150, as the guiding pin 152 moves within the first guide portion 168 (FIG. 9).

While locking dogs 228 are in the reset groove defined by the radially recessed surface 160 of the inner tube 104, further compressing the hold open rod 102 may translate the locking dogs 228 relative to the inner tube 104 to the intermediately radially recessed surface 162 until the locking dogs 228 reach the axially facing surface 280. When the locking dogs 228 are retained in the lock groove 226 and in contact with the axially facing surface 280, the locking dogs 228 may prevent the outer tube 106 from translating in the first axial direction D1 relative to the inner tube 104. The intermediately radially recessed surface 162 and the no-unlock feature 224 may interfere with the locking dogs 228 to prevent the locking dogs 228 from leaving the lock groove 226, without first unlocking the locking collar 108.

Thus, when compressed while in the first mode, the hold open rod 102 may be locked in the intermediate position. For example, the outer tube 106 may be prevented from translating in the first axial direction D1 relative to the inner tube 104 beyond the predetermined position (see e.g., FIGS. 13-15A).

The operator may unlock the locking collar 108 again to fully compress the hold open rod 102 (e.g., to fully close the door component 202). If the locking collar body 108a is retracted in the second axial direction D2 relative to the outer tube 106 and the locking pin 108b is pinned in the first portion 120 of the unlocking channel 106c, the hold open rod 102 may remain unlocked when fully compressed. In some embodiments, the locking collar may lock as the locking collar passes the first locking portion in a similar manner as when the locking collar passes the second locking portion while in the first mode. For example, the third recessed portion may be circumferentially offset from the first recessed portion so that the locking pin unpins when passing by the first locking portion.

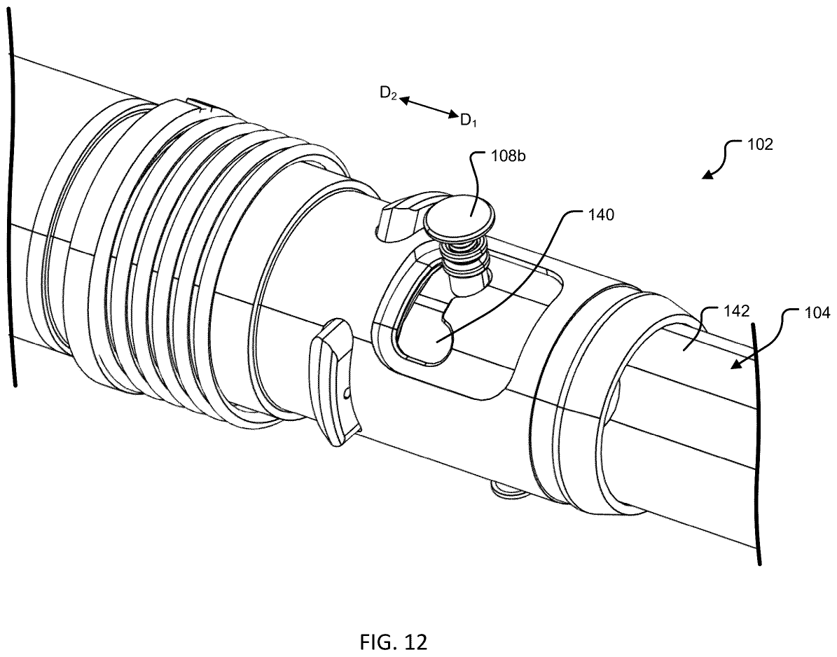

The locking collar 108 may be moved (e.g., rotated as discussed above) to the second unlocked position (e.g., from the first unlocked position while the hold open rod 102 is in the extended configuration), thereby transitioning the hold open rod 102 to the second mode. When the hold open rod 102 is in the second mode (see e.g., FIG. 12) and hold open rod 102 is in the extended configuration, compression of the hold open rod 102 to the intermediately extended configuration may not lock the hold open rod 102. For example, while pinned in the second portion 122 of the unlocking channel 106c, the locking pin 108b may translate in the first axial direction D1 along the second path defined by the second radially recessed portion 142. As the locking pin 108b translates in the first axial direction D1 to the first locking portion 104a, the first curved outer surface 126 may not unpin the locking pin 108b. Thus, the locking collar 108 may remain unlocked (e.g., in the second unlocked position, not the transitional unlock position) until reaching the first locking portion 104a.

The outer tube 106 may be translated, while the hold open rod 102 is in the second mode, relative to the inner tube 104 in the first axial direction D1 along the longitudinal axis X such that the locking collar 108 moves toward the first locking portion 104a. The locking collar 108 may remain unlocked such that the outer tube 106 translates relative to the inner tube 104 in the first axial direction D1 to and beyond the predetermined position (e.g., the axial position of the outer tube 106 relative to the inner tube 104, when the hold open rod 102 is in the intermediately extended configuration (see e.g., FIGS. 13-14)).

As the locking pin 108b translates in the first axial direction D1 past the first locking portion 104a, the locking pin 108b may unpin from the unlocking channel 106c by contacting the second curved outer surface 128 such that locking collar 108 transitions to a transitional unlock position. For example, the locking pin 108b may unpin from the second portion 122 of the unlocking channel 106c and then rotate in the second rotational direction R2 to re-pin into the first portion 120 of the unlocking channel 106c. The biasing member 230 may rotate in the second rotational direction R2 to re-pin into the first portion 120.

The second guide portion 170 of the guiding channel 150 (see e.g., FIG. 9) may initially constrain axial movement of the guiding pin 152 relative to the locking dogs 228 and the outer tube 106. Thus, axial movement of the locking collar body 108a in the first axial direction D1 relative to the unlocking channel 106c and/or the locking dogs 228 and the outer tube 106 may initially be prevented. Preventing the axial movement of the locking collar body 108a may prevent the locking dogs 228 from entering the lock groove 226.

Further compression of hold open rod 102 while the locking collar 108 is in the first unlocked position may result in the locking dogs 228 translating past the intermediately radially recessed surface 162 while retained in the unlock groove 222. For example, while the hold open rod 102 compresses such that the locking dogs 228 translate to the intermediately radially recessed surface 162, the first portion 120 of the unlocking channel 106c may continue to constrain axial movement of the locking pin 108b and/or the second guide portion 170 of the guiding channel 150 may continue to constrain axial movement of the guiding pin 152.

Once the locking dogs 228 translate to the intermediately radially recessed surface 162, the no-unlock feature 224 may be unable to translate in the first axial direction D1 relative to the locking dogs 228. Thus, the locking dogs 228 may be unable to enter the lock groove 226 until the locking dogs 228 move to the next reset groove (e.g., by extending the hold open rod 102 such that the locking dogs 228 move to the primary radially recessed surface 160).

The unpinning from the second portion 122 of the unlocking channel 106c may allow the biasing member 230 to move the locking collar body 108a relative to the locking dogs 228. For example, as the locking dogs 228 translate to the intermediately radially recessed surface 162 while the hold open rod 102 compresses, the biasing member 230 may rotate the locking collar body 108a relative to the outer tube 106 in the second rotational direction R2. For example, the biasing member 230 may rotate the locking collar body 108a relative to the outer tube 106 until the locking pin 108b becomes pinned in the first portion 120 of the unlocking channel 106c, as discussed above. When the locking pin 108b is received in the first portion 120, the guiding pin 152 may reach an intersection of the first guide portion 168 and the second guide portion 170.

Accordingly, the locking collar 108 may be unlocked and pinned in a similar manner as the first unlocked position after translating relative to the first locking portion 104a in the first axial direction D1. In some embodiments, the locking collar may be unlocked and pinned in a third unlocked position in which the locking pin is pinned in a different position (e.g., at a third portion of the locking channel).

As discussed above, the biasing member 230 may be unable to axially translate the locking collar body 108a in the first direction relative to the locking dogs 228 while the locking pin 108b remains pinned in the first portion 120. Thus, the hold open rod 102 may be compressed to its fully compressed configuration while the locking collar 108 remains unlocked and pinned in the first portion 120. In some embodiments, the inner tube includes one or more additional locking portions with a respective reset groove. Thus, in such embodiments, the hold open rod may be further compressed and locked at the next locking portion.

As discussed above, as the door component 202 closes, the hold open rod 102 may initially compress beyond its intermediately extended configuration (e.g., to its fully compressed configuration) and then expand to its stowed configuration. The locking pin 108b may translate along the third radially recessed portion 144 in the first axial direction D1 while pinned in the first portion 120. The locking pin 108b may also remain pinned in the first portion 120 while translating along the third radially recessed portion 144 in the second axial direction D2.

Thus, as the hold open rod 102 compresses toward its fully compressed and then extends to its stowed configuration (e.g., while the door component 202 closes), the locking pin 108b may remain pinned in the first portion 120, thereby maintaining the hold open rod 102 unlocked.

To again lock the hold open rod 102, an operator may unpin the locking pin 108b extending the hold open rod 102 until the locking pin 108b unpins and the locking dogs 228 reach the next reset groove. For example, the hold open rod 102 may be extended (e.g., by opening the door component 202) such that the locking pin 108b translates relative to the inner tube 104 in the second axial direction D2 and reaches the first curved outer surface 126, thereby causing the locking pin 108b to unpin. Unpinning the locking pin 108b transitions the locking collar 108 to its transitional unlocked position. While in the transitional unlocked position, the locking collar 108 remains unlocked until the locking dogs 228 reach the next reset groove (e.g., by extending the hold open rod 102 such that the locking dogs 228 move to the primary radially recessed surface 164). For example, interference between the locking dogs 228 and the no-unlock feature 224 and interference between the locking dogs 228 and the intermediately radially recessed surface 166 or the first curved outer surface 126 may prevent the locking dogs 228 from moving radially inwardly enough for the no-unlock feature 224 to move past the locking dogs 228 in the first axial direction D1.

In an embodiment, the operator may unpin the locking member by moving the locking pin radially outward from the first portion in a different manner (e.g., by manually retracting the movable member of the locking pin).

Thus, locking collar body 108a may be unable to move in the first axial direction D1 relative to the outer tube 106. For example, the guiding pin 152 may be unable to translate through the first guide portion 168 in the first axial direction D1 relative to the outer tube 106.

Upon reaching the next reset groove (e.g., by extending the hold open rod 102 such that the locking dogs 228 move to the primary radially recessed surface 164), the biasing member 230 may move the locking collar body 108a in the first axial direction D1 relative to the locking dogs 228 such that the locking dogs 228 are received in the lock groove 226. For example, the no-unlock feature 224 may translate in the first axial direction D1 past the locking dogs 228 by moving the locking dogs 228 radially inwardly toward the primary radially recessed surface 164.

The movement may include the biasing member 230 axially translating the locking collar body 108a relative to the locking dogs 228 and the outer tube 106 in the first axial direction D1 such that the locking dogs 228 enter the lock groove 226. The movement of the locking collar body 108a may follow a path defined by the guiding channel 150, as the guiding pin 152 moves within the first guide portion 168.

While the locking dogs 228 are in the lock groove 226, further compressing the hold open rod 102 may translate the locking dogs 228 relative to the inner tube 104 to the intermediately radially recessed surface 166 until the locking dogs 228 reach the axially facing surface 270. When the locking dogs 228 are retained in the lock groove 226 and in contact with the axially facing surface 270, the locking dogs 228 may prevent the outer tube 106 from translating in the first axial direction D1 relative to the inner tube 104. The intermediately radially recessed surface 166 and the no-unlock feature 224 may interfere with the locking dogs 228 to prevent the locking dogs 228 from leaving the lock groove 226, without first unlocking the locking collar 108.

As discussed above, the above-described movement of components of the hold open rod 102 may be relative to other components of the hold open rod 102, as opposed to movement in three-dimensional space. Thus, for example, when the outer tube 106 moves relative to the inner tube 104 in the first axial direction D1 the inner tube 104 moves relative to the outer tube 106 in the second axial direction D2, even if only the inner tube 104 translates in three-dimensional space along the second axial direction D2 when the door component 202 opens and closes.

Certain terminology is used in the following description for convenience only and is not limiting. The words “front,” “rear”, “right”, “left”, “lower” and “upper” designate directions in the drawings to which reference is made. The words “inner”, “internal”, and “interior” refer to directions towards the geometric center of the hold open rod 102, while the words “outer”, “external”, and “exterior” refer to directions away from the geometric center of the hold open rod 102. The terminology includes the above-listed words, derivatives thereof and words of similar import.

The following are a number of nonlimiting EXAMPLES of aspects of the disclosure.

In one general aspect, a hold open rod may include an outer tube. A hold open rod may also include an inner tube, where the inner tube and the outer tube are configured to translate relative to one another when the hold open rod is unlocked such that the inner tube translates within the outer tube along a longitudinal axis. A hold open rod may furthermore include a locking collar that is configured to lock and unlock the outer tube and the inner tube, where the locking collar is movable to a first unlocked position and a second unlocked position, where the hold open rod is configured to be in a first mode when the locking collar is in the first unlocked position, and where the hold open rod is configured to be in a second mode when the locking collar is in the second unlocked position. A hold open rod may in addition include where when in the first mode, the locking collar is configured to lock the outer tube and the inner tube when the outer tube translates relative to the inner tube in a first axial direction such that the locking collar moves to a first locking portion of the inner tube and prevents the outer tube from translating relative to the inner tube in the first axial direction beyond a predetermined position, and when in the second mode, the locking collar is configured to not lock the outer tube and the inner tube when the outer tube translates relative to the inner tube in the first axial direction to the first locking portion of the inner tube such that the locking collar allows the outer tube to translate relative to the inner tube to and beyond the predetermined position. Other embodiments of this aspect include corresponding apparatus configured to perform the actions of the methods.

The described implementations may also include one or more of the following features. The hold open rod where when in an initial locked position, the locking collar is configured to lock the outer tube at a second locking portion of the inner tube, that is offset from the first locking portion in a second axial direction that is opposite the first axial direction, such that the locking collar prevents the outer tube from translating relative to the inner tube in the first axial direction beyond an initial position that is offset from the predetermined position in the second direction. The hold open rod where when in the first mode, the locking collar is configured to allow the outer tube to move in the first axial direction relative to the inner tube beyond the initial position, and is configured to engage the first locking portion such that the outer tube and the inner tube are locked when the outer tube reaches the predetermined position, and when in the second mode, the locking collar is configured to allow the outer tube to move in the first axial direction relative to the inner tube beyond the initial position, and is configured to not engage the first locking portion when the outer tube reaches the predetermined position, where the outer tube and the inner tube remain unlocked thereby allowing the outer tube to move in the first axial direction relative to the inner tube beyond the predetermined position. The hold open rod where when the hold open rod is in the first mode the locking collar is configured to allow the outer tube and the inner tube to compress to a first compressed configuration and prevent the outer tube and the inner tube from compressing beyond the first compressed configuration, in which the outer tube and the inner tube together have a first length, and when in the second mode the locking collar is configured to allow the outer tube and the inner tube to compress to the first compressed configuration and compress beyond the first compressed configuration, in which the outer tube and the inner tube together have a second length that is less than the first length. The hold open rod where when locked, the locking collar is configured to prevent further compression of the outer tube and the inner tube, and is configured to allow extension of the outer tube and the inner tube. The hold open rod where the locking collar is configured to move between the first unlocked position and the second unlocked position such that the locking collar transitions the hold open rod between the first mode and the second mode. The hold open rod where the locking collar is configured to lock the outer tube relative to the inner tube when in an extended configuration. The hold open rod where the locking collar is rotatably movable between the first unlocked position and the second unlocked position. The hold open rod where the outer tube includes a first channel configured to receive a locking pin that is coupled to a locking collar body of the locking collar. The hold open rod where the first channel is U-shaped or bean shaped. The hold open rod where when in the first mode and the outer tube travels relative to the inner tube in the first axial direction, the locking pin is configured to be disposed within the first channel and to translate with the outer tube along a recessed portion of the inner tube to a radially outward surface of the inner tube that is configured to urge the locking pin radially outward into an unpinned position, thereby transitioning the locking collar from the first unlocked position to a first transitional unlocked and unpinned position. The hold open rod where the outer tube includes a guide channel, where the guide channel is configured to receive a radially inwardly extending guide member of the locking collar. The hold open rod where the first locking portion of the inner tube includes a first circumferentially extending groove that is axially offset from a second locking portion of the inner tube that includes a second circumferentially extending groove. The hold open rod where the locking collar includes one or more locking elements that are configured to move radially inwardly such that the one or more locking elements are disposed within the first circumferentially extending groove when the outer tube is locked in an intermediately extended configuration, and the one or more locking elements are configured to move radially inwardly such that the one or more locking elements are disposed within the second circumferentially extending groove when the outer tube is locked in an extended configuration in which the hold open rod has a length greater than when in the intermediately extended configuration. The hold open rod may include a biasing member that is configured to urge the locking collar in the first axial direction relative to the outer tube. The hold open rod where the biasing member is configured to circumferentially bias the locking collar into the first unlocked position. The hold open rod where the outer tube includes where the outer tube includes: a first elongated tubular body that circumscribes a majority of the inner tube when the hold open rod is in a compressed configuration; and a second elongated tubular body that is coupled an inner end of the first elongated tubular body and circumscribes a portion of the inner tube that is offset from the majority of the inner tube when the hold open rod is in the compressed configuration. Implementations of the described techniques may include hardware, a method, or a process.

Some implementations herein relate to a method. For example, a method of operating a hold open rod may include moving a locking collar, which is movable to a first unlocked position and movable to a second unlocked position, to the second unlocked position, where the hold open rod is in a first mode when the locking collar is in the first unlocked position, and where the hold open rod is in a second mode when the locking collar is in the second unlocked position. A method of operating a hold open rod may also include translating, while the hold open rod is in the second mode, the outer tube relative to the inner tube in a first axial direction along a longitudinal axis such that the locking collar moves toward a first locking portion of the inner tube, where hold open rod remains unlocked such that the outer tube translates relative to the inner tube in the first axial direction to and beyond a predetermined position. A method of operating a hold open rod may furthermore include where the locking collar is configured to, when in the first mode, lock the outer tube and the inner tube when the outer tube translates relative to the inner tube in the first axial direction such that the locking collar moves to the first locking portion of the inner tube and prevents the outer tube from translating relative to the inner tube in the first axial direction beyond the predetermined position. Other embodiments of this aspect include corresponding apparatus configured to perform the actions of the methods.

The described implementations may also include one or more of the following features. The method of where the moving the locking collar step includes moving the locking collar from the first unlocked position to the second unlocked position, such that the hold open rod transitions from the first mode to the second mode. The method where the translating, while the hold open rod is in the second mode, step comprises the locking collar not locking the outer tube and the inner tube, where the locking collar allows the outer tube to translate relative to the inner tube to and beyond the predetermined position. The method may include translating, while the hold open rod is in the first mode, the outer tube relative to the inner tube in the first axial direction along the longitudinal axis to the first locking position of the inner tube such that the locking collar prevents the outer tube from moving relative to the inner tube beyond the predetermined position in the first axial direction. The method where the translating, while the hold open rod is in the first mode, step comprises translating the outer tube relative to the first tube in the first axial direction until the locking collar locks the outer tube and the inner tube by engaging the first locking portion of the inner tube such that the locking collar prevents the outer tube from moving relative to the inner tube beyond the predetermined position in the first axial direction. The method where the locking collar is moved to the first unlocked position and the inner tube translates within the outer tube along the longitudinal axis, while the locking collar is in the first unlocked position, until the locking collar prevents the outer tube from moving beyond the predetermined position in the first axial direction relative to the inner tube. The method where preventing the outer tube from moving beyond the predetermined position comprises the locking collar preventing the outer tube and the inner tube from compressing while the outer tube is in the predetermined position. The method where translating the outer tube relative to the inner tube in the first axial direction comprises the outer tube remaining translationally fixed to a stationary portion of a vehicle while the inner tube moves with a door of the vehicle in the second axial direction to translate along the longitudinal axis. The method where moving the locking collar to the second unlocked position comprises inserting a locking pin of the locking collar into a first channel of the outer tube such that the locking pin is in a bypass pinned position such that the locking collar is in the second unlocked position. The method where moving the locking collar to the to the first unlocked position comprises inserting the locking pin of the locking collar into the first channel of the outer tube such that the locking pin is in a primary pinned position such that the locking collar is in the first unlocked position. The method may include translating the outer tube relative to the inner tube, while in the first mode, in the first axial direction, which comprises moving the locking pin in the first axial direction toward a first circumferentially extending groove of the first locking portion of the inner tube, along a first recessed portion of the outer tube, to a radially outward surface of the outer tube that urges the locking pin radially outward out of the first channel into an unpinned position, thereby transitioning the locking collar from the first unlocked position to the first transitional unlocked and unpinned position prior to reaching the first locking portion. The method where translating the outer tube relative to the inner tube, while in the second mode, in the first axial direction comprises translating the locking pin toward the first circumferentially extending groove along a second recessed portion of the outer tube, and after passing the first circumferentially extending groove, contacting a second radially outward surface of the outer tube that urges the locking pin radially outward out of a portion of the first channel, thereby transitioning the locking collar from the second unlocked position. The method where moving the locking collar to the first unlocked position or the second unlocked position comprises moving a guide member of the locking collar within a guiding channel of the outer tube. The method where the guide member moves axially within a first guide portion of the guiding channel to an intersection of the first guide portion and a second guide portion of the guiding channel when moving the locking collar to the first unlocked position. The method where the guide member moves circumferentially within the second guide portion away from the first guide portion of the guiding channel when moving the locking collar to the second unlocked position. The method where the translating the outer tube relative to the inner tube comprises compressing the hold open rod. The method where the translating the outer tube relative to the inner tube comprises compressing the hold open rod to an intermediate position. The method where moving the locking collar to the second unlocked position comprises moving the locking collar from the first unlocked position to the second unlocked position. The method where moving the locking collar comprises rotating the locking collar from the first unlocked position to the second unlocked position. The method may include locking, with the locking collar, the outer tube, and the inner tube in an extended configuration. Implementations of the described techniques may include hardware, a method, or a process.

Although the disclosure has been described in detail, it should be understood that various changes, substitutions, and alterations can be made herein without departing from the spirit and scope of the invention as defined by the appended claims. Additionally, any of the embodiments disclosed herein can incorporate features disclosed with respect to any of the other embodiments disclosed herein. Moreover, the scope of the present disclosure is not intended to be limited to the particular embodiments described in the specification. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

1. A hold open rod, comprising:
an outer tube;
an inner tube, wherein the inner tube and the outer tube are configured to translate relative to one another when the hold open rod is unlocked such that the inner tube translates within the outer tube along a longitudinal axis; and
a locking collar that is configured to lock and unlock the outer tube and the inner tube, wherein the locking collar is movable to a first unlocked position and a second unlocked position, wherein the hold open rod is configured to be in a first mode when the locking collar is in the first unlocked position, and wherein the hold open rod is configured to be in a second mode when the locking collar is in the second unlocked position;
wherein when in the first mode, the locking collar is configured to lock the outer tube and the inner tube when the outer tube translates relative to the inner tube in a first axial direction such that the locking collar moves to a first locking portion of the inner tube and prevents the outer tube from translating relative to the inner tube in the first axial direction beyond a predetermined position, and when in the second mode, the locking collar is configured to not lock the outer tube and the inner tube when the outer tube translates relative to the inner tube in the first axial direction to the first locking portion of the inner tube such that the locking collar allows the outer tube to translate relative to the inner tube to and beyond the predetermined position.

2. The hold open rod of claim 1, wherein when in an initial locked position, the locking collar is configured to lock the outer tube at a second locking portion of the inner tube, that is offset from the first locking portion in a second axial direction that is opposite the first axial direction, such that the locking collar prevents the outer tube from translating relative to the inner tube in the first axial direction beyond an initial position that is offset from the predetermined position in the second axial direction.

3. The hold open rod of claim 2, wherein when in the first mode, the locking collar is configured to allow the outer tube to move in the first axial direction relative to the inner tube beyond the initial position, and is configured to engage the first locking portion such that the outer tube and the inner tube are locked when the outer tube reaches the predetermined position, and when in the second mode, the locking collar is configured to allow the outer tube to move in the first axial direction relative to the inner tube beyond the initial position, and is configured to not engage the first locking portion when the outer tube reaches the predetermined position, whereby the outer tube and the inner tube remain unlocked thereby allowing the outer tube to move in the first axial direction relative to the inner tube beyond the predetermined position.

4. The hold open rod of claim 2, wherein when the hold open rod is in the first mode the locking collar is configured to allow the outer tube and the inner tube to compress to a first compressed configuration and prevent the outer tube and the inner tube from compressing beyond the first compressed configuration, in which the outer tube and the inner tube together have a first length, and when in the second mode the locking collar is configured to allow the outer tube and the inner tube to compress to the first compressed configuration and compress beyond the first compressed configuration, in which the outer tube and the inner tube together have a second length that is less than the first length.

5. The hold open rod of claim 4, wherein when locked, the locking collar is configured to prevent further compression of the outer tube and the inner tube, and is configured to allow extension of the outer tube and the inner tube.

6. (canceled)

7. The hold open rod of claim 1, wherein the locking collar is configured to lock the outer tube relative to the inner tube when in an extended configuration.

8. (canceled)

9. The hold open rod of claim 1, wherein the outer tube includes a first channel configured to receive a locking pin that is coupled to a locking collar body of the locking collar.

10-12. (canceled)

13. The hold open rod of claim 1, wherein the first locking portion of the inner tube includes a first circumferentially extending groove that is axially offset from a second locking portion of the inner tube that includes a second circumferentially extending groove; and
wherein the locking collar includes one or more locking elements that are configured to move radially inwardly such that the one or more locking elements are disposed within the first circumferentially extending groove when the outer tube is locked in an intermediately extended configuration, and the one or more locking elements are configured to move radially inwardly such that the one or more locking elements are disposed within the second circumferentially extending groove when the outer tube is locked in an extended configuration in which the hold open rod has a length greater than when in the intermediately extended configuration.

14. (canceled)

15. The hold open rod of claim 1, further comprising a biasing member that is configured to urge the locking collar in the first axial direction relative to the outer tube.

16. (canceled)

17. The hold open rod of claim 1, wherein the outer tube includes:
a first elongated tubular body that circumscribes a majority of the inner tube when the hold open rod is in a compressed configuration; and
a second elongated tubular body that is coupled an inner end of the first elongated tubular body and circumscribes a portion of the inner tube that is offset from the majority of the inner tube when the hold open rod is in the compressed configuration.

18. A method of operating a hold open rod, comprising:
moving a locking collar, which is movable to a first unlocked position and movable to a second unlocked position, to the second unlocked position, wherein the hold open rod is in a first mode when the locking collar is in the first unlocked position, and wherein the hold open rod is in a second mode when the locking collar is in the second unlocked position; and
translating, while the hold open rod is in the second mode, an outer tube relative to an inner tube in a first axial direction along a longitudinal axis such that the locking collar moves toward a first locking portion of the inner tube, wherein hold open rod remains unlocked such that the outer tube translates relative to the inner tube in the first axial direction to and beyond a predetermined position;
wherein the locking collar is configured to, when in the first mode, lock the outer tube and the inner tube when the outer tube translates relative to the inner tube in the first axial direction such that the locking collar moves to the first locking portion of the inner tube and prevents the outer tube from translating relative to the inner tube in the first axial direction beyond the predetermined position.

19. The method of claim 18, wherein the moving the locking collar step includes moving the locking collar from the first unlocked position to the second unlocked position, such that the hold open rod transitions from the first mode to the second mode.

20. The method of claim 18, wherein the translating, while the hold open rod is in the second mode, step comprises the locking collar not locking the outer tube and the inner tube, whereby the locking collar allows the outer tube to translate relative to the inner tube to and beyond the predetermined position.

21. The method of claim 18, further comprising translating, while the hold open rod is in the first mode, the outer tube relative to the inner tube in the first axial direction along the longitudinal axis to the first locking portion of the inner tube such that the locking collar prevents the outer tube from moving relative to the inner tube beyond the predetermined position in the first axial direction.

22. The method of claim 21, wherein the translating, while the hold open rod is in the first mode, step comprises translating the outer tube relative to the inner tube in the first axial direction until the locking collar locks the outer tube and the inner tube by engaging the first locking portion of the inner tube such that the locking collar prevents the outer tube from moving relative to the inner tube beyond the predetermined position in the first axial direction.

23-24. (canceled)

25. The method of claim 18, wherein translating the outer tube relative to the inner tube in the first axial direction comprises the outer tube remaining translationally fixed to a stationary portion of a vehicle while the inner tube moves with a door of the vehicle in a second axial direction to translate along the longitudinal axis.

26. The method of claim 18, wherein moving the locking collar to the second unlocked position comprises inserting a locking pin of the locking collar into a first channel of the outer tube such that the locking pin is in a bypass pinned position such that the locking collar is in the second unlocked position.

27. (canceled)

28. The method of claim 26, further comprising translating the outer tube relative to the inner tube, while in the first mode, in the first axial direction, which comprises moving the locking pin in the first axial direction toward a first circumferentially extending groove of the first locking portion of the inner tube, along a first recessed portion of the outer tube, to a radially outward surface of the outer tube that urges the locking pin radially outward out of the first channel into an unpinned position, thereby transitioning the locking collar from the first unlocked position to a first transitional unlocked and unpinned position prior to reaching the first locking portion.

29. (canceled)

30. The method of claim 18, wherein moving the locking collar to the first unlocked position or the second unlocked position comprises moving a guide member of the locking collar within a guiding channel of the outer tube.

31-32. (canceled)

33. The method of claim 18, wherein the translating the outer tube relative to the inner tube comprises compressing the hold open rod.

34-37. (canceled)
